(19) United States
(12) Patent Application Publication
(10) Pub. No.: US 2017/0266192 A1
(43) Pub. Date: Sep. 21, 2017

(54) METHODS FOR IMPROVING DIAPHRAGM FUNCTION

(71) Applicant: CYTOKINETICS, INC., South San Francisco, CA (US)

(72) Inventors: Jeffrey R. JASPER, South San Francisco, CA (US); Fady MALIK, South San Francisco, CA (US); Darren T. HWEE, South San Francisco, CA (US)

(21) Appl. No.: 15/444,063

(22) Filed: Feb. 27, 2017

Related U.S. Application Data
(63) Continuation of application No. 14/389,805, filed on Oct. 1, 2014, now abandoned.
(60) Provisional application No. 61/619,261, filed on Apr. 2, 2012.

(51) Int. Cl. A61K 31/52; A61K 31/505; A61K 31/506; A61K 31/4985

(52) U.S. Cl. CPC A61K 31/52; A61K 31/4985; A61K 31/506; A61K 31/505

(57) ABSTRACT

Provided are compositions and methods for improving diaphragm function in a patient. In some embodiments, the methods comprise administering to a patient or contacting a diaphragm skeletal muscle fiber with an effective amount of a skeletal muscle troponin activator or pharmaceutically acceptable salt thereof. Likewise, compositions and methods are also provided for increasing the function, activity, efficiency, sensitivity to calcium, or time to fatigue of skeletal muscle in the diaphragm. In some embodiments, the patient receiving such administration suffers from diaphragmatic atrophy.

This application claims priority to U.S. Appln. No. 61/619,261, filed Apr. 2, 2012, which is incorporated herein by reference for all purposes.

Diaphragm separates the thoracic and abdominal cavities and is the principal muscle of respiration. Diaphragm is primarily composed of fatigue-resistant slow-switch type I and fast-switch type IIa myofibers. Disease processes that interfere with diaphragmatic innervation, contractile properties, or mechanical coupling to the chest wall can result in diaphragmatic dysfunction which, in turn, can lead to dyspnea, decreased exercise performance, sleep-disordered breathing, constitutional symptoms, hypersomnia, reduced quality of life, atelectasis, and respiratory failure.

Dysfunction of the diaphragm ranges from a partial loss of the ability to generate pressure (weakness) to a complete loss of diaphragmatic function (paralysis). Patients with bilateral diaphragmatic paralysis or severe diaphragmatic weakness are likely to have dyspnea or recurrent respiratory failure. They can have considerable dyspnea at rest, when supine, with exertion, or when immersed in water above their waist. Further, patients with bilateral diaphragmatic paralysis are at an increased risk for sleep fragmentation and hypoventilation during sleep. Initial symptoms may include fatigue, hypersomnia, depression, morning headaches, and frequent nocturnal awakenings. Other complications of bilateral diaphragmatic paralysis include subsegmental atelectasis and infections of the lower respiratory tract.

Diaphragm dysfunction can be caused and coexist with other diseases or conditions such as amyotrophic lateral sclerosis (ALS), chronic obstructive pulmonary disease (COPD), asthma, heart failure, spinal muscular atrophy (SMA), and muscular dystrophy.

In healthy humans most skeletal muscles are composed of both fast and slow fibers, although the proportions of each vary with muscle type. Slow skeletal fibers, often called type I fibers, have more structural similarity with cardiac muscle and tend to be used more for fine and postural control. They usually have a greater oxidative capacity and are more resistant to fatigue with continued use. Fast skeletal muscle fibers, often called type II fibers, are classified into fast oxidative (IIa) and fast glycolytic (type IIx/d) fibers. While these muscle fibers have different myosin types, they share many components including the troponin and tropomyosin regulatory proteins. Fast skeletal muscle fibers tend to exert greater force but fatigue faster than slow skeletal muscle fibers and are functionally useful for acute, large scale movements such as rising from a chair or correcting falls. Healthy diaphragm contains approximately equal amounts of fast and slow skeletal muscle fibers, but the proportion can change under diseased conditions.

Provided are compositions and methods for improving diaphragm function. In some embodiments, the methods comprise administering to a patient or contacting a diaphragm skeletal muscle fiber with an effective amount of a skeletal muscle troponin activator. Likewise, compositions and methods are also provided for increasing the function, activity, efficiency, sensitivity to calcium, or time to fatigue of skeletal muscle in the diaphragm.

In some embodiments, the patient receiving such administration suffers from diaphragmatic atrophy. In some embodiments, the patient suffers from a disease or condition selected from ventilator-induced diaphragmatic weakness or atrophy, steroid-induced diaphragmatic atrophy, hemidiaphragm paralysis, fetal hydrops, pleural effusion, botulinum poisoning, organophosphate poisoning, Guillain-Barre syndrome, phrenic nerve dysfunction, asthma, heart failure, amyotrophic lateral sclerosis (ALS), spinal muscular atrophy (SMA), and muscular dystrophy. In some embodiments, the patient is in use of mechanical ventilation. In some embodiments, the patient undertakes an intense physical activity or is in an environment with a reduced partial pressure of oxygen in the air.

In some embodiments, the skeletal muscle troponin activator is a chemical entity selected from compounds of Formula A and compounds of Formula B:

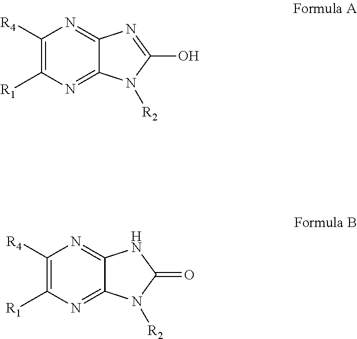

and pharmaceutically acceptable salts thereof, wherein R₁, R₂ and R₄ are as defined herein.

In some embodiments, the skeletal muscle troponin activator is a chemical entity selected from compounds of Formula I:

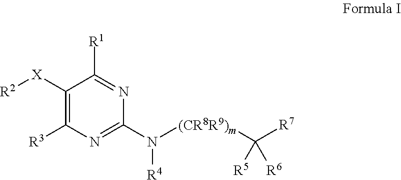

or a pharmaceutically acceptable salt thereof, wherein R¹, R², R³, R⁴, R⁵, R⁶, R⁷, R⁸, R⁹, X and m are as defined herein.

Other aspects and embodiments will be apparent to those skilled in the art from the following detailed description.

### BRIEF DESCRIPTION OF THE FIGURES

FIG. 1 shows concentration-response curves for Compound A in skinned rabbit psoas fiber and skinned rat diaphragm fiber preparations at a constant calcium concentration.

FIG. 2 shows the force produced by skinned rat diaphragm fibers at various calcium concentrations when treated with Compound B at different concentrations.

FIG. 3 shows the force produced by skinned rat diaphragm fibers at various calcium concentrations when treated with Compound C at different concentrations.

FIG. 4A shows mean diaphragm cross sectional area from SHAM and LAD rats. Mean diaphragm cross sectional area was significantly lower in HF diaphragm muscle.

FIG. 4B shows mean diaphragm type I myofiber area cross sectional area from SHAM and LAD rats.

FIG. 4C shows mean diaphragm type IIa myofiber area cross sectional area from SHAM and LAD rats. Significant atrophy can be seen in type IIa fibers in HF diaphragms.

FIG. 4D shows mean diaphragm type IIb/x myofiber area cross sectional area from SHAM and LAD rats. Significant atrophy can be seen in type IIb/x fibers in HF diaphragms.

FIG. 5 shows the force production in SHAM and HF rat diaphragm muscle measured by ex-vivo electrical field stimulation. HF diaphragm muscle produced significantly lower force compared to SHAM diaphragms.

FIG. 6 shows force production in rat diaphragm muscle measured by ex-vivo electrical field stimulation in the presence and absence of Compound B. Diaphragm muscle treated with Compound B produced significantly more force compared to vehicle-only diaphragms at frequencies up to 30 Hz of electrical stimulation.

FIG. 7 shows force production measured over 600 contractions in rat diaphragm muscle ex vivo by field electrical stimulation in the presence and absence of Compound B. Diaphragm muscle treated with Compound B produced significantly more force compared to vehicle-only diaphragms in a dose-dependent manner.

FIG. 8A shows force production in SHAM rat diaphragm muscle measured by ex-vivo electrical field stimulation in the presence and absence of Compound D. Compound D significantly increased force in SHAM diaphragms at submaximal frequencies of electrical stimulation.

FIG. 8B shows force production in LAD rat diaphragm muscle measured by ex-vivo electrical field stimulation in the presence and absence of Compound D. Compound D significantly increased force in LAD diaphragms at submaximal frequencies of electrical stimulation.

FIG. 9 shows force produced by LAD and SHAM skinned rat diaphragm fibers at various calcium concentrations in the presence and absence of Compound D. Compound D significantly increased Ca²⁺ sensitivity in both SHAM and HF diaphragm fibers.

FIG. 10 shows force production measured ex vivo by electrical field stimulation in mouse diaphragms harvested from WT and SOD1 mice at various concentrations of Compound C. Both WT and SOD1 diaphragm muscle treated with Compound C produced significantly more force compared to vehicle-only diaphragms at frequencies up to 30 Hz of electrical stimulation.

FIG. 11 shows respiratory parameters assessed before, during, and after a 30 minute 5% CO₂ challenge by unrestrained whole body plethysmography in SOD1 mice. Compared to vehicle-treated animals, Compound C treated animals had significantly higher tidal volume at baseline and at recovery after a 30 minute exposure to a 5% CO₂ gas mixture.

As used in the present specification, the following words and phrases are generally intended to have the meanings as set forth below, except to the extent that the context in which they are used indicates otherwise.

Throughout this application, unless the context indicates otherwise, references to a compound of a formula, e.g., Formula A or I, includes all subgroups of the formula defined herein, including all substructures, subgenera, preferences, embodiments, examples and particular compounds described herein.

References to a compound of a formula and subgroups thereof include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. “Crystalline form,” “polymorph,” and “novel form” may be used interchangeably herein, and are meant to include all crystalline and amorphous forms of the compound, including, for example, polymorphs, pseudopolymorphs, solvates (including hydrates), co-crystals, unsolvated polymorphs (including anhydrates), conformational polymorphs, and amorphous forms, as well as mixtures thereof, unless a particular crystalline or amorphous form is referred to. In some embodiments, references to a compound of a formula (e.g., a compound of Formula A, Formula B, and/or Formula I) and subgroups thereof include polymorphs, solvates, co-crystals, isomers, tautomers and/or oxides thereof. In some embodiments, references to a compound of a formula (e.g., a compound of Formula A, Formula B, and/or Formula I) and subgroups thereof include polymorphs, solvates, and/or co-crystals thereof. In some embodiments, references to a compound of a formula (e.g., a compound of Formula A, Formula B, and/or Formula I) and subgroups thereof include isomers, tautomers and/or oxides thereof. In some embodiments, references to a compound of a formula (e.g., a compound of Formula A, Formula B, and/or Formula I) and subgroups thereof include solvates thereof. Similarly, the term “salts” includes solvates of salts of compounds.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable.

When a range of values is given (e.g., C₁₋₆ alkyl), each value within the range as well as all intervening ranges are included. For example, “C₁₋₆ alkyl” includes C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

When a moiety is defined as being optionally substituted, it may be substituted as itself or as part of another moiety. For example, if R^{x} is defined as “C₁₋₆ alkyl or OC₁₋₆ alkyl, wherein C₁₋₆ alkyl is optionally substituted with halogen”, then both the C₁₋₆ alkyl group alone and the C₁₋₆ alkyl that makes up part of the OC₁₋₆ alkyl group may be substituted with halogen.

“Alkyl” encompasses straight chain and branched chain having the indicated number of carbon atoms, usually from 1 to 20 carbon atoms, for example 1 to 8 carbon atoms, such as 1 to 6 carbon atoms. For example C₁-C₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “butyl” is meant to include n-butyl, sec-butyl, isobutyl and t-butyl; “propyl” includes npropyl and isopropyl. “Lower alkyl” refers to alkyl groups having one to seven carbons. In certain embodiments, “lower alkyl” refers to alkyl groups having one to six carbons. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is a subset of alkyl, referring to the same residues as alkyl, but having two points of attachment. Alkylene groups will usually have from 2 to 20 carbon atoms, for example 2 to 8 carbon atoms, such as from 2 to 6 carbon atoms. For example, C₀ alkylene indicates a covalent bond and C₁ alkylene is a methylene group.

“Haloalkyl” includes straight and branched carbon chains having the indicated number of carbon atoms (e.g., 1 to 6 carbon atoms) substituted with at least one halogen atom. In instances wherein the haloalkyl group contains more than one halogen atom, the halogens may be the same (e.g., dichloromethyl) or different (e.g., chlorofluoromethyl). Examples of haloalkyl groups include, but are not limited to, chloromethyl, dichloromethyl, trichloromethyl, fluoromethyl, difluoromethyl, trifluoromethyl, chlorofluoromethyl, 2-fluoroethyl, 2,2-difluoroethyl, 2,2,2-trifluoroethyl, 1,2-difluoroethyl, 2-chloroethyl, 2,2-dichloroethyl, 2,2,2-trichloroethyl, 1,2-dichloroethyl, pentachloroethyl, and pentafluoroethyl.

“Alkenyl” refers to an unsaturated branched or straight-chain alkyl group having at least one carbon-carbon double bond derived by the removal of one molecule of hydrogen from adjacent carbon atoms of the parent alkyl. The group may be in either the cis or trans configuration about the double bond(s). Typical alkenyl groups include, but are not limited to, ethenyl; propenyls such as prop-1-en-1-yl, prop-1-en-2-yl, prop-2en-1-yl (allyl), prop-2-en-2-yl; butenyls such as but-1-en-1-yl, but-1-en-2-yl, 2-methylprop-1-en-1-yl, but-2-en-1-yl, but-2-en-1-yl, but-2-en-2-yl, buta-1,3-dien-1-yl, buta-1,3dien-2-yl; and the like. In certain embodiments, an alkenyl group has from 2 to 20 carbon atoms and in other embodiments, from 2 to 6 carbon atoms. “Lower alkenyl” refers to alkenyl groups having two to six carbons.

“Alkynyl” refers to an unsaturated branched or straight-chain alkyl group having at least one carbon-carbon triple bond derived by the removal of two molecules of hydrogen from adjacent carbon atoms of the parent alkyl. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms and in other embodiments, from 3 to 6 carbon atoms. “Lower alkynyl” refers to alkynyl groups having two to six carbons.

“Cycloalkyl” indicates a non-aromatic carbocyclic ring, usually having from 3 to 7 ring carbon atoms. The ring may be saturated or have one or more carbon-carbon double bonds. Examples of cycloalkyl groups include cyclopropyl, cyclobutyl, cyclopentyl, cyclopentenyl, cyclohexyl, and cyclohexenyl, as well as bridged and caged ring groups such as norbornane.

“Cycloalkenyl” indicates a non-aromatic carbocyclic ring, containing the indicated number of carbon atoms (e.g., 3 to 10, or 3 to 8, or 3 to 6 ring carbon atoms) and at least one carbon-carbon double bond derived by the removal of one molecule of hydrogen from adjacent carbon atoms of the corresponding cycloalkyl. Cycloalkenyl groups may be monocyclic or polycyclic (e.g., bicyclic, tricyclic). Examples of cycloalkenyl groups include cyclopropenyl, cyclobutenyl, cyclopentenyl, cyclopentadienyl, and cyclohexenyl, as well as bridged and caged ring groups (e.g., bicyclo[2.2.2]octene). In addition, one ring of a polycyclic cycloalkenyl group may be aromatic, provided the polycyclic alkenyl group is bound to the parent structure via a non-aromatic carbon atom. For example, inden-1-yl (wherein the moiety is bound to the parent structure via a non-aromatic carbon atom) is considered a cycloalkenyl group, while inden-4-yl (wherein the moiety is bound to the parent structure via an aromatic carbon atom) is not considered a cycloalkenyl group. Examples of polycyclic cycloalkenyl groups consisting of a cycloalkenyl group fused to an aromatic ring are described below.

The term “alkoxy” refers to the group —O-alkyl, including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons.

The term “substituted alkoxy” refers to alkoxy wherein the alkyl constituent is substituted (i.e., —O-(substituted alkyl)) wherein “substituted alkyl” refers to alkyl wherein one or more (such as up to 5, for example, up to 3) hydrogen atoms are replaced by a substituent independently chosen from: —R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO²R^{a}, —OCONR^{b}R^{c}, —OCONR^{b}R^{c}, —OP(O)(OR^{b})OR^{c}, sulfanyl (such as SR^{b}), sulfinyl (such as —SOR^{a}), and sulfonyl (such as —SO²R^{a} and —SO₂NR^{b}R^{c}),

where R^{a} is chosen from optionally substituted C₁-C₆ alkyl, optionally substituted alkenyl, optionally substituted alkynyl, optionally substituted aryl, and optionally substituted heteroaryl;

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄alkyl)C(O)(C₁-C₄alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

In some embodiments, a substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of 2-20, such as 2-10, and for example, 2-5. Another substituted alkoxy group is hydroxyalkoxy or —OCH₂(CH₂)_{y}OH, where y is an integer of 1-10, such as 1-4.

The term “alkoxycarbonyl” refers to a group of the formula (alkoxy)(C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality and wherein substituted refers to alkyl wherein one or more (such as up to 5, for example, up to 3) hydrogen atoms are replaced by a substituent independently chosen from:

—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}) optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OCONR^{b}R^{c}, —OP(O)(OR^{b})OR^{c}, sulfanyl (such as SR^{b}), sulfinyl (such as —SOR^{a}), and sulfonyl (such as —SO₂R^{a} and —SO₂NR^{b}R^{c}),

where R^{a} is chosen from optionally substituted C₁-C₆ alkyl, optionally substituted alkenyl, optionally substituted alkynyl, optionally substituted aryl, and optionally substituted heteroaryl;

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

“Aryl” encompasses:

6-membered carbocyclic aromatic rings, for example, benzene;

bicyclic ring systems wherein at least one ring is carbocyclic and aromatic, for example, naphthalene, indane, and tetralin; and

tricyclic ring systems wherein at least one ring is carbocyclic and aromatic, for example, fluorene.

For example, aryl includes 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

“Aralkoxy” refers to the group —O-aralkyl. Similarly, “heteroaralkoxy” refers to the group —O-heteroaralkyl; “aryloxy” refers to —O-aryl; and “heteroaryloxy” refers to the group —O-heteroaryl.

“Aralkyl” refers to a residue in which an aryl moiety is attached to the parent structure via an alkyl residue. Examples include benzyl, phenethyl, phenylvinyl, phenylallyl and the like. “Heteroaralkyl” refers to a residue in which a heteroaryl moiety is attached to the parent structure via an alkyl residue. Examples include furanylmethyl, pyridinylmethyl, pyrimidinylethyl and the like.

“Halogen” or “halo” refers to fluorine, chlorine, bromine or iodine. Dihaloaryl, dihaloalkyl, trihaloaryl etc. refer to aryl and alkyl substituted with a plurality of halogens, but not necessarily a plurality of the same halogen; thus 4-chloro-3-fluorophenyl is within the scope of dihaloaryl.

“Heteroaryl” encompasses:

5- to 7-membered aromatic, monocyclic rings containing one or more, for example, from 1 to 4, or in certain embodiments, from 1 to 3, heteroatoms chosen from N, O, and S, with the remaining ring atoms being carbon;

bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring; and

tricyclic heterocycloalkyl rings containing one or more, for example, from 1 to 5, or in certain embodiments, from 1 to 4, heteroatoms chosen from N, O, and S, with the remaining ring atoms being carbon and wherein at least one heteroatom is present in an aromatic ring.

For example, heteroaryl includes a 5- to 7-membered heterocycloalkyl, aromatic ring fused to a 5- to 7-membered cycloalkyl or heterocycloalkyl ring. For such fused, bicyclic heteroaryl ring systems wherein only one of the rings contains one or more heteroatoms, the point of attachment may be at either ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridazinyl, triazolyl, quinolinyl, pyrazolyl, and 5,6,7,8-tetrahydroisoquinolinyl. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl, cycloalkyl, or heterocycloalkyl, as defined herein

Substituted heteroaryl also includes ring systems substituted with one or more oxide (—O⁻) substituents, such as pyridinyl N-oxides.

By “heterocycloalkyl” is meant a single, non-aromatic ring, usually with 3 to 7 ring atoms, containing at least 2 carbon atoms in addition to 1-3 heteroatoms independently selected from oxygen, sulfur, and nitrogen, as well as combinations comprising at least one of the foregoing heteroatoms. The ring may be saturated or have one or more carbon-carbon double bonds. Suitable heterocycloalkyl groups include, for example (as numbered from the linkage position assigned priority 1), 2-pyrrolidinyl, 2,4-imidazolidinyl, 2,3-pyrazolidinyl, 2-piperidyl, 3-piperidyl, 4-piperidyl, and 2,5-piperizinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo (═O) or oxide (—O⁻) substituents, such as piperidinyl N-oxide, morpholinyl-N-oxide, 1-oxo-1-thiomorpholinyl and 1,1-dioxo-1-thiomorpholinyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteratoms independently selected from oxygen, sulfur, and nitrogen and is not aromatic.

“Heterocycloalkenyl” indicates a non-aromatic ring having the indicated number of atoms (e.g., 3 to 10, or 3 to 7, membered heterocycloalkyl) made up of one or more heteroatoms (e.g., 1, 2, 3 or 4 heteroatoms) selected from N, O and S and with the remaining ring atoms being carbon, and at least one double bond derived by the removal of one molecule of hydrogen from adjacent carbon atoms, adjacent nitrogen atoms, or adjacent carbon and nitrogen atoms of the corresponding heterocycloalkyl. Heterocycloalkenyl groups may be monocyclic or polycyclic (e.g., bicyclic, tricyclic). When nitrogen is present in a heterocycloalkenyl ring, it may, where the nature of the adjacent atoms and groups permits, exist in an oxidized state (i.e., N⁺—O⁻). Additionally, when sulfur is present in a heterocycloalkenyl ring, it may, where the nature of the adjacent atoms and groups permits, exist in an oxidized state (i.e., S⁺—O⁻ or —SO₂—). Examples of heterocycloalkenyl groups include dihydrofuranyl (e.g., 2,3-dihydrofuranyl, 2,5-dihydrofuranyl), dihydrothiophenyl (e.g., 2,3-dihydrothiophenyl, 2,5-dihydrothiophenyl), dihydropyrrolyl (e.g., 2,3-dihydro-1H-pyrrolyl, 2,5-dihydro-1H-pyrrolyl), dihydroimidazolyl (e.g., 2,3-dihydro-1H-imidazolyl, 4,5-dihydro-1H-imidazolyl), pyranyl, dihydropyranyl (e.g., 3,4-dihydro-2H-pyranyl, 3,6-dihydro-2H-pyranyl), tetrahydropyridinyl (e.g., 1,2,3,4-tetrahydropyridinyl, 1,2,3,6-tetrahydropyridinyl) and dihydropyridine (e.g., 1,2-dihydropyridine, 1,4-dihydropyridine). In addition, one ring of a polycyclic heterocycloalkenyl group may be aromatic (e.g., aryl or heteroaryl), provided the polycyclic heterocycloalkenyl group is bound to the parent structure via a non-aromatic carbon or nitrogen atom. For example, a 1,2-dihydroquinolin-1-yl group (wherein the moiety is bound to the parent structure via a non-aromatic nitrogen atom) is considered a heterocycloalkenyl group, while 1,2-dihydroquinolin-8-yl group (wherein the moiety is bound to the parent structure via an aromatic carbon atom) is not considered a heterocycloalkenyl group. Examples of polycyclic heterocycloalkenyl groups consisting of a heterocycloalkenyl group fused to an aromatic ring are described below.

Examples of polycyclic rings consisting of an aromatic ring (e.g., aryl or heteroaryl) fused to a non-aromatic ring (e.g., cycloalkyl, cycloalkenyl, heterocycloalkyl, heterocycloalkenyl) include indenyl, 2,3-dihydro-1H-indenyl, 1,2,3,4-tetrahydronaphthalenyl, benzo[1,3]dioxolyl, tetrahydroquinolinyl, 2,3-dihydrobenzo[1,4]dioxinyl, indolinyl, isoindolinyl, 2,3-dihydro-1H-indazolyl, 2,3-dihydro-1H-benzo[d]imidazolyl, 2,3-dihydrobenzofuranyl, 1,3-dihydroisobenzofuranyl, 1,3-dihydrobenzo[c]isoxazolyl, 2,3-dihydrobenzo[d]isoxazolyl, 2,3-dihydrobenzo[d]oxazolyl, 2,3-dihydrobenzo[b]thiophenyl, 1,3-dihydrobenzo[c]thiophenyl, 1,3-dihydrobenzo[c]isothiazolyl, 2,3-dihydrobenzo[d]isothiazolyl, 2,3-dihydrobenzo[d]thiazolyl, 5,6-dihydro-4H-cyclopenta[d]thiazolyl, 4,5,6, 7-tetrahydrobenzo[d]thiazolyl, 5,6-dihydro-4H-pyrrolo[3,4-d]thiazolyl, 4,5,6,7-tetrahydrothiazolo[5,4-c]pyridinyl, indolin-2-one, indolin-3-one, isoindolin-1-one, 1,2-dihydroindazol-3-one, 1H-benzo[d]imidazol-2(3H)-one, benzofuran-2(3H)-one, benzofuran-3(2H)-one, isobenzofuran-1(3H)-one, benzo[c]isoxazol-3(1H)-one, benzo[d]isoxazol-3(2H)-one, benzo[d]oxazol-2(3H)-one, benzo[b]thiophen-2(3H)-one, benzo[b]thiophen-3(2H)-one, benzo[c]thiophen-1 (3H)-one, benzo[c]isothiazol-3(1H)-one, benzo[d]isothiazol-3(2H)-one, benzo[d]thiazol-2(3H)-one, 4,5-dihydropyrrolo[3,4-d]thiazol-6-one, 1,2-dihydropyrazolo[3,4-d]thiazol-3-one, quinolin-4(3H)-one, quinazolin-4(3H)-one, quinazoline-2,4(1H,3H)-dione, quinoxalin-2(1H)-one, quinoxaline-2,3(1H,4H)-dione, cinnolin-4(3H)-one, pyridin-2(1H)-one, pyrimidin-2(1H)-one, pyrimidin-4(3H)-one, pyridazin-3(2H)-one, 1H-pyrrolo[3,2-b]pyridin-2(3H)-one, 1H-pyrrolo[3,2-c]pyridin-2(3H)-one, 1H-pyrrolo[2,3-c]pyridin-2(3H)-one, 1H-pyrrolo[2,3-b]pyridin-2(3H)-one, 1,2-dihydropyrazolo[3,4-d]thiazol-3-one and 4,5-dihydropyrrolo[3,4-d]thiazol-6-one. As discussed herein, whether each ring is considered an aryl, heteroaryl, cycloalkyl, cycloalkenyl, heterocycloalkyl or heterocycloalkenyl group is determined by the atom through which the moiety is bound to the parent structure.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(.±.)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R)- or (S)-. The present invention is meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

The stereochemistry depicted in the structures of cyclic meso compounds is not absolute; rather the stereochemistry is intended to indicate the positioning of the substituents relative to one another, e.g., cis or trans. For example,

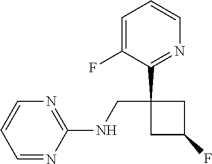 [SMILES: Fc1cccnc1[C@]1(CNc2ncccn2)C[C@H](F)C1]

is intended to designate a compound wherein the fluorine and pyridyl substituents on the cyclobutyl ring are in a cis configuration to one another, while

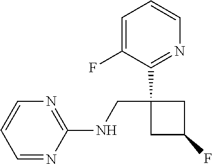 [SMILES: Fc1cccnc1[C@]1(CNc2ncccn2)C[C@@H](F)C1]

is intended to designate a compound wherein the fluorine and pyridyl substituents on the cyclobutyl ring are in a trans configuration to one another.

When a compound can exist as one or more meso isomers, all possible meso isomers are intended to be included. For example, the compound {[3-fluoro-1-(3-fluoro(2-pyridyl))cyclobutyl]methyl}pyrimidin-2-ylamine is intended to include both cis and trans meso isomers:

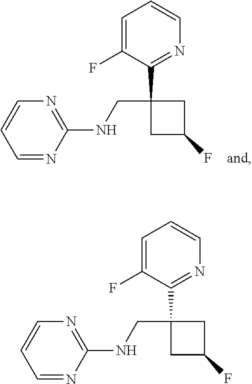 [SMILES: Fc1cccnc1[C@]1(CNc2ncccn2)C[C@H](F)C1 ; Fc1cccnc1[C@]1(CNc2ncccn2)C[C@@H](F)C1]

and mixtures thereof. Unless otherwise indicated, compounds described herein include all possible meso isomers and mixtures thereof.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconverision of pentane-2,4-dione and 4-hydroxypent-3-en-2-one tautomers. Another example of tautomerization is phenol-keto tautomerization. A specific example of phenol-keto tautomerization is the interconverision of pyridin-4-ol and pyridin-4(1H)-one tautomers. Compounds of certain of the disclosed formulas are tautomeric.

A leaving group or atom is any group or atom that will, under the reaction conditions, cleave from the starting material, thus promoting reaction at a specified site. Suitable examples of such groups include, but are not limited to, halogen atoms, mesyloxy, p-nitrobenzensulphonyloxy and tosyloxy groups.

Protecting group has the meaning conventionally associated with it in organic synthesis, i.e. a group that selectively blocks one or more reactive sites in a multifunctional compound such that a chemical reaction can be carried out selectively on another unprotected reactive site and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999). For example, a hydroxy protected form is where at least one of the hydroxy groups present in a compound is protected with a hydroxy protecting group. Likewise, amines and other reactive groups may similarly be protected.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts that retain the biological effectiveness and properties of the compounds described herein and, which are not biologically or otherwise undesirable. In many cases, the compounds described herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “solvate” refers to a compound (e.g., a compound selected from Formula A or I, or a pharmaceutically acceptable salt thereof) in physical association with one or more molecules of a pharmaceutically acceptable solvent. It will be understood that “a compound of Formula X” encompass the compound of Formula X, and solvates of those compounds, as well as mixtures thereof.

A “chelate” is formed by the coordination of a compound to a metal ion at two (or more) points. The term “compound” is intended to include chelates of compounds. Similarly, “salts” includes chelates of salts and “solvates” includes chelates of solvates.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound”.

The term “prodrug” refers to a substance administered in an inactive or less active form that is then transformed (e.g., by metabolic processing of the prodrug in the body) into an active compound. The rationale behind administering a prodrug is to optimize absorption, distribution, metabolism, and/or excretion of the drug. Prodrugs may be obtained by making a derivative of an active compound (e.g., a compound of Formula A or another compound described herein) that will undergo a transformation under the conditions of use (e.g., within the body) to form the active compound. The transformation of the prodrug to the active compound may proceed spontaneously (e.g., by way of a hydrolysis reaction) or it can be catalyzed or induced by another agent (e.g., an enzyme, light, acid or base, and/or temperature). The agent may be endogenous to the conditions of use (e.g., an enzyme present in the cells to which the prodrug is administered, or the acidic conditions of the stomach) or the agent may be supplied exogenously. Prodrugs can be obtained by converting one or more functional groups in the active compound into another functional group, which is then converted back to the original functional group when administered to the body. For example, a hydroxyl functional group can be converted to a sulfonate, phosphate, ester or carbonate group, which in turn can be hydrolyzed in vivo back to the hydroxyl group. Similarly, an amino functional group can be converted, for example, into an amide, carbamate, imine, urea, phosphenyl, phosphoryl or sulfenyl functional group, which can be hydrolyzed in vivo back to the amino group. A carboxyl functional group can be converted, for example, into an ester (including silyl esters and thioesters), amide or hydrazide functional group, which can be hydrolyzed in vivo back to the carboxyl group. Examples of prodrugs include, but are not limited to, phosphate, acetate, formate and benzoate derivatives of functional groups (such as alcohol or amine groups) present in the compounds of Formula A and other compounds described herein.

The compounds described herein can be enriched isotopic forms, e.g., enriched in the content of ²H, ³H, ¹¹C, ¹³C and/or ¹⁴C. In some embodiments, the compound contains at least one deuterium atom. Such deuterated forms can be made, for example, by the procedure described in U.S. Pat. Nos. 5,846,514 and 6,334,997. Such deuterated compounds may improve the efficacy and increase the duration of action of compounds described herein. Deuterium substituted compounds can be synthesized using various methods, such as those described in: Dean, D., Recent Advances in the Synthesis and Applications of Radiolabeled Compounds for Drug Discovery and Development, Curr. Pharm. Des., 2000; 6(10); Kabalka, G. et al., The Synthesis of Radiolabeled Compounds via Organometallic Intermediates, Tetrahedron, 1989, 45(21), 6601-21; and Evans, E., Synthesis of radiolabeled compounds, J. Radioanal. Chem., 1981, 64(1-2), 9-32.

The terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl wherein one or more (such as up to 5, for example, up to 3) hydrogen atoms are replaced by a substituent independently chosen from:

—R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substitutent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OCONR^{b}R^{c}, —OP(O)(OR^{b})OR^{c}, sulfanyl (such as SR^{b}), sulfinyl (such as —SOR^{a}), and sulfonyl (such as —SO₂R^{a} and —SO₂NR^{b}R^{c}),

where

R^{a} is chosen from optionally substituted C₁-C₆ alkyl, optionally substituted cycloalkyl, optionally substituted heterocycloalkyl, optionally substituted alkenyl, optionally substituted alkynyl, optionally substituted aryl, and optionally substituted heteroaryl;

R^{b} is chosen from hydrogen, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “sulfanyl” refers to the groups: —S-(optionally substituted alkyl), —S-(optionally substituted cycloalkyl), —S-(optionally substituted aryl), —S-(optionally substituted heteroaryl), and —S-(optionally substituted heterocycloalkyl).

The term “sulfinyl” refers to the groups: —S(O)—H, —S(O)-(optionally substituted alkyl), —S(O)-(optionally substituted cycloalkyl), —S(O)-(optionally substituted amino), —S(O)-(optionally substituted aryl), —S(O)-(optionally substituted heteroaryl), and —S(O)-(optionally substituted heterocycloalkyl).

The term “sulfonyl” refers to the groups: —S(O₂)—H, —S(O₂)-(optionally substituted alkyl), —S(O₂)-(optionally substituted cycloalkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

The term “active agent” is used to indicate a compound that has biological activity. In some embodiments, an “active agent” is a compound having therapeutic utility. In some embodiments, the compound enhances at least one aspect of skeletal muscle function or activity, such as power output, skeletal muscle force, skeletal muscle endurance, oxygen consumption, efficiency, and/or calcium sensitivity.

Compounds also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

Chemical entities include, but are not limited to, compounds of the disclosed formulas, and all pharmaceutically acceptable forms thereof. Pharmaceutically acceptable forms of the compounds recited herein include pharmaceutically acceptable salts, chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, chelates, non-covalent complexes, prodrugs, and mixtures.

The terms “patient” and “subject” refer to an animal, such as a mammal bird or fish. In some embodiments, the patient or subject is a mammal. Mammals include, for example, mice, rats, dogs, cats, pigs, sheep, horses, cows and humans. In some embodiments, the patient or subject is a human, for example a human that has been or will be the object of treatment, observation or experiment. The compounds, compositions and methods described herein can be useful in both human therapy and veterinary applications.

As used herein, “skeletal muscle” includes skeletal muscle tissue as well as components thereof, such as skeletal muscle fibers, the myofibrils comprising the skeletal muscle fibers, the skeletal sarcomere which comprises the myofibrils, and the various components of the skeletal sarcomere described herein, including skeletal myosin, actin, tropomyosin, troponin C, troponin I, troponin T and fragments and isoforms thereof. In some embodiments, “skeletal muscle” includes fast skeletal muscle tissue as well as components thereof, such as fast skeletal muscle fibers, the myofibrils comprising the fast skeletal muscle fibers, the fast skeletal sarcomere which comprises the myofibrils, and the various components of the fast skeletal sarcomere described herein, including fast skeletal myosin, actin, tropomyosin, troponin C, troponin I, troponin T and fragments and isoforms thereof. Skeletal muscle does not include cardiac muscle or a combination of sarcomeric components that occurs in such combination in its entirety in cardiac muscle.

As used herein, the term “therapeutic” refers to the ability to modulate the contractility of fast skeletal muscle. As used herein, “modulation” (and related terms, such as “modulate”, “modulated”, “modulating”) refers to a change in function or efficiency of one or more components of the fast skeletal muscle sarcomere, including myosin, actin, tropomyosin, troponin C, troponin I, and troponin T from fast skeletal muscle, including fragments and isoforms thereof, as a direct or indirect response to the presence of a compound described herein, relative to the activity of the fast skeletal sarcomere in the absence of the compound. The change may be an increase in activity (potentiation) or a decrease in activity (inhibition), and may be due to the direct interaction of the compound with the sarcomere, or due to the interaction of the compound with one or more other factors that in turn affect the sarcomere or one or more of its components. In some embodiments, modulation is a potentiation of function or efficiency of one or more components of the fast skeletal muscle sarcomere, including myosin, actin, tropomyosin, troponin C, troponin I, and troponin T from fast skeletal muscle, including fragments and isoforms thereof. Modulation may be mediated by any mechanism and at any physiological level, for example, through sensitization of the fast skeletal sarcomere to contraction at lower Ca²⁺ concentrations. As used herein, “efficiency” or “muscle efficiency” means the ratio of mechanical work output to the total metabolic cost.

The term “therapeutically effective amount” or “effective amount” refers to that amount of a compound selected from the disclosed formulas that is sufficient to effect treatment, as defined below, when administered to a mammal in need of such treatment. The therapeutically effective amount will vary depending upon the subject and disease condition being treated, the weight and age of the subject, the severity of the disease condition, the particular compound selected from the disclosed formulas, the dosing regimen to be followed, timing of administration, the manner of administration and the like, all of which can readily be determined by one of ordinary skill in the art.

“Treatment” or “treating” means any treatment of a disease in a patient, including:
(a) preventing the disease, that is, causing the clinical symptoms of the disease not to develop;
(b) inhibiting the disease;
(c) slowing or arresting the development of clinical symptoms; and/or
(d) relieving the disease, that is, causing the regression of clinical symptoms.

As used herein, “power output” of a muscle means work/cycle time and may be scaled up from PoLo/cycle time units based on the properties of the muscle. Power output may be modulated by changing, for example, activating parameters during cyclical length changes, including timing of activation (phase of activation) and the period of activation (duty cycle.)

“ATPase” refers to an enzyme that hydrolyzes ATP. ATPases include proteins comprising molecular motors such as the myosins.

As used herein, “selective binding” or “selectively binding” refers to preferential binding to a target protein in one type of muscle or muscle fiber as opposed to other types. For example, a compound selectively binds to fast skeletal troponin C if the compound preferentially binds troponin C in the troponin complex of a fast skeletal muscle fiber or sarcomere in comparison with troponin C in the troponin complex of a slow muscle fiber or sarcomere or with troponin C in the troponin complex of a cardiac sarcomere.

Provided are skeletal muscle troponin activators that can effectively improve the function of diaphragm, in particular diaphragm with dysfunction. Dysfunction of the diaphragm can include a partial loss of the ability to generate pressure (weakness) and a complete loss of diaphragmatic function (paralysis). Such improvement is particularly useful, clinically, when the diaphragm is under stress or suffering dysfunction, such as in the face of neuromuscular disorders and/or conditions marked by muscle weakness.

It is contemplated that skeletal muscle troponin activators, in particular those disclosed herein, selectively sensitize fast skeletal muscle in the diaphragm to calcium by binding to its troponin complex. By increasing the calcium sensitivity of the troponin-tropomyosin regulatory complex, which is the calcium sensor within the sarcomere that regulates the actin-myosin force-generating interaction, the skeletal muscle troponin activators improve muscle force generation. As a consequence of their activity on the troponin-tropomyosin complex, the skeletal muscle troponin activators amplify the response of muscle to neuromuscular input and also decrease the fatigability of muscle.

Provided are compositions and methods for improving diaphragm function. In some embodiments, the methods entail administering to a patient or contacting a diaphragm skeletal muscle fiber with an effective amount of a skeletal muscle troponin activator. Compositions and methods are also provided for increasing the function, activity, efficiency, sensitivity to calcium, or time to fatigue of skeletal muscle in the diaphragm. In some embodiments, the skeletal muscle in the diaphragm is fast skeletal muscle.

In some embodiments, the skeletal muscle troponin activator is administered to a patient in need of improving diaphragm function. In some embodiments, the patient suffers from diaphragm dysfunction. In some embodiments, the patient suffers from diaphragmatic weakness or paralysis. In some embodiments, the patient suffers from unilateral or bilateral diaphragmatic weakness or paralysis.

Many diseases and conditions are known to cause or coexist with diaphragm dysfunction, or diaphragm weakness or paralysis. Non-limiting examples of such diseases and conditions include multiple sclerosis, stroke, Arnold-Chiari malformation, quadriplegia, amyotrophic lateral sclerosis (ALS), poliomyelitis, spinal muscular atrophy (SMA), syringomyelia, Guillain-Barre syndrome, tumor compression, neuralgic neuropathy, critical-illness polyneuropathy, chronic inflammatory demyelinating polyneuropathy, Charcot-Marie-Tooth disease, idiopathic, hyperinflation including chronic obstructive pulmonary disease (COPD) and asthma, myasthenia gravis, Lambert-Eaton syndrome, botulism, organophosphate exposure, drug use, muscular dystrophies (including Duchenne muscular dystrophy, Becker muscular dystrophy, limb-girdle muscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy), myositis (infectious, inflammatory, metabolic), acid maltase deficiency, glucocorticoids, and disuse atrophy.

Provided are methods of treating patients suffering from diaphragm dysfunction caused by, or also suffering from, any one or more of these diseases or conditions.

In some embodiments, the patient suffers from a disease or condition selected from sleep-disordered breathing, ventilator-induced diaphragmatic weakness or atrophy, steroid-induced diaphragmatic atrophy, hemidiaphragm paralysis, fetal hydrops, pleural effusion, botulinum poisoning, organophosphate poisoning, Guillain-Barre syndrome, phrenic nerve dysfunction and asthma.

In some embodiments, the patient suffers from diaphragmatic atrophy. Diaphragmatic atrophy, for instance, can be caused by disuse. In some embodiments, the patient is in use of mechanical ventilation. The combination of complete diaphragm inactivity and mechanical ventilation can elicit disuse atrophy of myofibers. It is contemplated that compounds described herein can improve diaphragm function or treat or prevent diaphragmatic atrophy in patients undergoing a mechanical ventilation treatment.

Exercise in patients with congestive heart failure is often limited by fatigue and shortness of breath (dyspnea). Importantly, fast (type 2) skeletal muscle fibers appear to atrophy in the diaphragm (Howell et al. J Appl Physiol. 1995 August; 79(2):389-97). An increase in diaphragmatic function caused by administration of fast skeletal troponin activators as described herein will increase respiratory function and improve symptoms of dyspnea and increase capacity for physical activity in heart failure patients. In some embodiments, the method comprises improving diaphragm function of a heart failure patient by administering a fast skeletal muscle troponin activator.

A primary cause of morbidity and mortality in patients with ALS is due to respiratory failure. By improving diaphragmatic and respiratory function by administration of fast skeletal troponin activators, ALS patient quality of life will be improved. In some embodiments, the method comprises improving diaphragm function of a patient suffering from ALS by administering a fast skeletal muscle troponin activator.

Muscular Dystrophy is a group of muscle diseases that weaken the musculoskeletal system and hamper locomotion. Muscular dystrophies are characterized by progressive skeletal muscle weakness, defects in muscle proteins, and the death of muscle cells and tissue. Types of muscular dystrophies include Duchenne muscular dystrophy, Becker muscular dystrophy, limb-girdle muscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy. In some embodiments the method comprises improving diaphragm function of a patient suffering from muscular dystrophy by administering a fast skeletal muscle troponin activator. In some embodiments, the muscular dystrophy is selected from Duchenne muscular dystrophy, Becker muscular dystrophy, limb-girdle muscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy.

The methods described herein, in some embodiments, can also benefit healthy individuals. For instance, individuals that undertake intense physical activities or individuals in an environment with a reduced partial pressure of oxygen in the air (e.g., at high elevation), can also benefit from treatment with a skeletal muscle troponin activator.

In some embodiments, in addition to or instead of improving diaphragm function in a subject, administration of the skeletal muscle troponin activator improves the function of one or more other muscles involved in respiration, such as external intercostal muscle or internal intercostal muscle.

Patients in need to improving diaphragm function can be identified with methods known in the art. Chest radiographs, for instance, may reveal elevated hemidiaphragms and basal subsegmental atelectasis. Further, fluoroscopy of the diaphragm has been extensively used to evaluate diaphragmatic function.

Pulmonary-function tests, especially measurements of upright and supine vital capacity, are noninvasive tests of diaphragmatic function. With unilateral diaphragmatic paralysis, total lung capacity may be mildly restricted (70 to 79% of the predicted value). In severe diaphragmatic weakness or bilateral diaphragmatic paralysis, there is typically moderate-to-severe restriction (30 to 50% of the predicted value for total lung capacity). In both unilateral and bilateral diaphragmatic paralysis, the restrictive dysfunction becomes more severe when the patient is in the supine position. A decrease in vital capacity of 30 to 50% when the patient is supine supports the diagnosis of bilateral diaphragmatic paralysis, whereas a decrease in vital capacity of 10 to 30% of the vital capacity when the patient is seated may be seen with mild diaphragmatic weakness or unilateral diaphragmatic paralysis. In some embodiments, the patient has unilateral diaphragmatic paralysis. In some embodiments, the patient has severe diaphragmatic weakness or bilateral diaphragmatic paralysis.

In some embodiments, the patient has a forced vital capacity (FVC) lower than about 75%, or alternatively lower than about 70%, 65%, 60%, 55%, 50%, 45%, 40%, 35%, 30%, 25% or 20% of predicted of healthy individual in similar conditions. In some embodiments, the patient shows evidence of increased work of breathing indicative of reduced diaphragm function, e.g., significant tachypnea, intercostal retractions, or other physical signs of respiratory distress thought to be.

Two additional measures of diaphragmatic function are maximal static inspiratory pressure and sniff nasal inspiratory pressure. In some embodiments, the patient has a maximal static inspiratory pressure or sniff nasal inspiratory pressure that is lower than about 75%, or alternatively lower than about 70%, 65%, 60%, 55%, 50%, 45%, 40%, 35%, 30%, 25% or 20% of predicted of healthy individual in similar conditions.

Direct measures of diaphragmatic function include invasive methods such as transdiaphragmatic pressure [Pdi] or noninvasive means such as ultrasonography. Here, a sniff Pdi or Pdi max greater than 80 cm of water in men and greater than 70 cm of water in women rules out clinically significant diaphragmatic weakness. A twitch Pdi greater than 10 cm of water with unilateral phrenic-nerve stimulation or greater than 20 cm of water with bilateral phrenic-nerve stimulation also rules out clinically significant weakness.

In some embodiments, the patient is a male patient having a sniff Pdi or Pdi max lower than about 80 cm of water, or alternatively lower than about 75 cm, 70 cm, 65 cm, 60 cm, 55 cm, 50 cm, 45 cm, 40 cm, 35 cm, 30 cm, or 25 cm of water. In some embodiments, the patient is a female patient having a sniff Pdi or Pdi max lower than about 70 cm of water, or alternatively lower than about 65 cm, 60 cm, 55 cm, 50 cm, 45 cm, 40 cm, 35 cm, 30 cm, 25 cm, or 20 cm of water. In some embodiments, the patient has a twitch Pdi lower than about 10 cm, or alternatively lower than about 9 cm, 8 cm, 7 cm, 6 cm, 5 cm, 4 cm, 3 cm, 2 cm or 1 cm of water with unilateral phrenic-nerve stimulation. In some embodiments, the patient has a twitch Pdi lower than about 20 cm, or alternatively lower than about 19 cm, 18 cm, 17 cm, 16 cm, 15 cm, 14 cm, 13 cm, 12 cm, 11 cm, 10 cm, 9 cm, 8 cm, 7 cm, 6 cm, 5 cm, 4 cm, 3 cm, 2 cm or 1 cm of water with bilateral phrenic-nerve stimulation.

In some embodiments, the methods for improving diaphragm function described herein further comprises administering to the patient a second therapeutic agent suitable for improving diaphragm function. Such second therapeutic agents, when employed in combination with the compounds and compositions described herein, may be used, for example, in those amounts indicated in the Physicians' Desk Reference (PDR) or as otherwise determined by one of ordinary skill in the art.

In some embodiments, a skeletal muscle troponin activate is a chemical entity chosen from compounds of Formula A and compounds of Formula B:

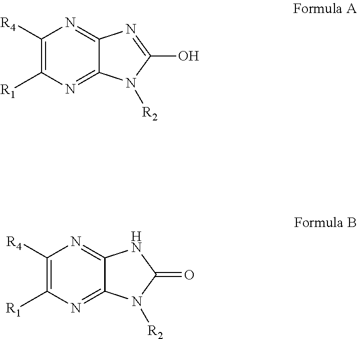

and pharmaceutically acceptable salts thereof, wherein

R₁ and R₄ are independently selected from hydrogen, halo, hydroxy, optionally substituted acyl, optionally substituted alkyl, optionally substituted amino, optionally substituted alkenyl, optionally substituted alkynyl, optionally substituted cycloalkyl, optionally substituted heteroaryl, optionally substituted alkoxy, optionally substituted aminocarbonyl, sulfonyl, sulfanyl, sulfinyl, carboxy, optionally substituted alkoxycarbonyl, and cyano; and in the alternative, R₄ and R₁, taken together with any intervening atoms, form a fused ring system selected from optionally substituted fused aryl, optionally substituted fused heteroaryl, optionally substituted fused cycloalkyl, and optionally substituted fused heterocycloalkyl; and

R₂ is selected from optionally substituted alkyl, optionally substituted cycloalkyl, optionally substituted aryl, optionally substituted heteroaryl, and optionally substituted heterocycloalkyl;

provided that

R₁ is not hex-1-enyl; and further provided that

the compound of Formula A or the compound of Formula B is not
(S)-6-bromo-1-(1-phenylethyl)-1H-imidazo[4,5-b]pyrazin-2(3H)-one;
1,5,6-trimethyl-1H-imidazo[4,5-b]pyrazin-2(3H)-one;
1-methyl-1H-imidazo[4,5-b]pyrazin-2(3H)-one;
6-bromo-1-(3-nitrobenzyl)-1H-imidazo[4,5-b]pyrazin-2(3H)-one;
5-(hydroxymethyl)-1,6-dimethyl-1H-imidazo[4,5-b]pyrazin-2(3H)-one; or
1-(piperidin-4-yl)-1H-imidazo[4, 5-b]pyrazin-2(3H)-one.

In some embodiments, R₂ is selected from optionally substituted lower alkyl, optionally substituted cycloalkyl, optionally substituted alkoxy, and optionally substituted heterocycloalkyl.

In some embodiments, R₂ is selected from heterocycloalkyl, cycloalkyl, lower alkyl, and lower alkyl substituted with optionally substituted phenyl, hydroxy, optionally substituted alkoxy, optionally substituted amino and optionally substituted heterocycloalkyl.

In some embodiments, R₂ is selected from 1-(R)-phenylethyl, 1-(S)-phenylethyl, benzyl, 3-pentyl, 4-heptyl, 4-methyl-1-morpholinopentan-2-yl isobutyl, cyclohexyl, cyclopropyl, sec-butyl, tert-butyl, isopropyl, 1-hydroxybutan-2-yl, tetra hydro-2H-pyran-4-yl, 1-methoxybutan-2-yl, 1-aminobutan-2-yl, and 1-morpholinobutan-2-yl.

In some embodiments, R₁ is selected from hydrogen, halo, acyl, optionally substituted lower alkyl, optionally substituted amino, optionally substituted pyrazolyl, optionally substituted alkenyl, optionally substituted alkynyl, optionally substituted lower alkoxy, and —S-(optionally substituted lower alkyl).

In some embodiments, R₁ is selected from hydrogen, halo, acyl, optionally substituted lower alkyl, dialkylamino, amino substituted with an alkyl group and with a group chosen from acyl, aminocarbonyl, alkoxycarbonyl, and sulfonyl; optionally substituted pyrazolyl, optionally substituted alkenyl, optionally substituted alkynyl, optionally substituted lower alkoxy, and —S-(optionally substituted lower alkyl).

In some embodiments, R₁ is selected from hydrogen, halo, acyl, alkenyl, alkynyl, lower alkoxy, optionally substituted amino, pyrazolyl substituted with lower alkyl, —S-(optionally substituted lower alkyl), lower alkyl, and lower alkyl substituted with halo.

In some embodiments, R₁ is selected from hydrogen, halo, acyl, alkenyl, alkynyl, lower alkoxy, dialkylamino, amino substituted with an alkyl group and with a group chosen from acyl, aminocarbonyl, alkoxycarbonyl, and sulfonyl, pyrazolyl substituted with lower alkyl, —S-(optionally substituted lower alkyl), lower alkyl, and lower alkyl substituted with halo.

In some embodiments, R₁ is selected from hydrogen, bromo, chloro, fluoro, methyl, ethyl, propyl, hexenyl, butenyl, propenyl, vinyl, ethynyl, methoxy, ethoxy, methylsulfanyl, dimethylamino, and methyl substituted with up to three fluoro groups.

In some embodiments, R₁ is selected from hydrogen, bromo, chloro, fluoro, methyl, ethyl, n-propyl, isopropyl, dimethylamino, isobuten-1-yl, (Z)-propen-1-yl, (E)-propen-1-yl, propen-2-yl, vinyl, ethynyl, methoxy, ethoxy, methylsulfanyl, and trifluoromethyl.

In some embodiments, R₄ is selected from hydrogen, halo, acyl, optionally substituted alkyl, alkenyl, optionally substituted cycloalkyl, optionally substituted aminocarbonyl, sulfanyl, optionally substituted amino, and optionally substituted alkoxycarbonyl.

In some embodiments, R₄ is selected from hydrogen, halo, acyl, optionally substituted lower alkyl, lower alkenyl, optionally substituted cycloalkyl, optionally substituted aminocarbonyl, sulfanyl, optionally substituted amino, and optionally substituted lower alkoxycarbonyl.

In some embodiments, R₄ is selected from hydrogen, halo, acyl, lower alkyl, lower alkenyl, cycloalkyl, optionally substituted aminocarbonyl, sulfanyl, and lower alkoxycarbonyl.

In some embodiments, R₄ is selected from hydrogen, bromo, chloro, fluoro, acetyl, methyl, ethyl, vinyl, cyclohexen-1-yl, methylcarbamoyl, dimethylcarbamoyl, methylsulfanyl, and methoxycarbonyl.

In some embodiments, R₄ is hydrogen.

In some embodiments, R₄ and R₁, taken together with any intervening atoms, form a fused ring system selected from optionally substituted fused aryl, optionally substituted fused cycloalkyl, and optionally substituted fused heterocycloalkyl.

In some embodiments, R₄ and R₁ are taken together to form an optionally substituted benzo group.

In some embodiments, R₄ and R₁ are taken together to form a benzo group.

In some embodiments, the skeletal muscle troponin activator is a chemical entity selected from compounds of Formula A and compounds of Formula B:

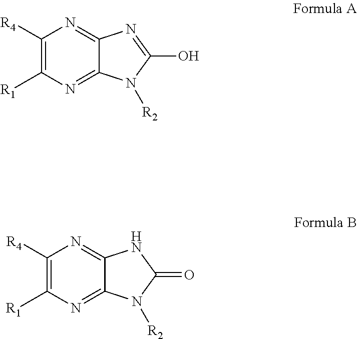

and pharmaceutically acceptable salts thereof, wherein:

R₁ is alkenyl or alkynyl;

R₄ is hydrogen; and

R₂ is selected from 3-pentyl, 4-heptyl, 4-methyl-1-morpholinopentan-2-yl isobutyl, cyclohexyl, cyclopropyl, sec-butyl, tert-butyl, isopropyl, 1-hydroxybutan-2yl, tetrahydro-2H-pyran-4-yl, 1-methoxybutan-2-yl, 1-aminobutan-2-yl, and 1-morpholinobutan-2-yl;
provided that R₁ is not hex-1-enyl.

In some embodiments, the compound of Formula A is chosen from:
1-((1R)-1-methyl-2-morpholin-4-ylethyl)-6-bromoimidazo[4, 5-b]pyrazin-2-ol;
1-(ethylpropyl)-6-ethynylimidazo[4,5-b]pyrazin-2-ol;
1-(ethylpropyl)-6-methoxyimidazo[4,5-b]pyrazin-2-ol;
1-(1,1-dim ethyl-2-morpholin-4-ylethyl)-6-bromoimidazo[4,5-b]pyrazin-2-ol;
6-(1H-1,2,3-triazol-4-yl)-1-(ethylpropyl)imidazo[4, 5-b]pyrazin-2-ol;
1-(ethylpropyl)-6-(trifluoromethyl)imidazo[4,5-b]pyrazin-2-ol;
1-[(1R)-1-(morpholin-4-ylmethyl)propyl]-6-ethynylimidazo[4, 5-b]pyrazin-2-ol;
1-(ethylpropyl)-6-{2-[1-(ethylpropyl)-2-hydroxyimidazo[4,5-e]pyrazin-6-yl]ethynyl}imidazo[4, 5-b]pyrazin-2-ol;
6-(dimethylamino)-1-(ethylpropyl)imidazo[4,5-b]pyrazin-2-ol;
6-ethyl-1-(ethylpropyl)imidazo[4,5-b]pyrazin-2-ol;
(E)-1-(pentan-3-yl)-6-(prop-1-enyl)-1H-imidazo[4,5-b]pyrazin-2-ol;
(E)-1-cyclohexyl-6-(prop-1-enyl)-1H-imidazo[4,5-b]pyrazin-2-ol;
(E)-1-cyclopropyl-6-(prop-1-enyl)-1H-imidazo[4,5-b]pyrazin-2-ol;
(E)-1-isopropyl-6-(prop-1-enyl)-1H-imidazo[4,5-b]pyrazin-2-ol;
(E)-6-(prop-1-enyl)-1-(tetrahydro-2H-pyran-4-yl)-1H-imidazo[4,5-b]pyrazin-2-ol;
(R)-6-(methylthio)-1-(1-phenylethyl)-1H-imidazo[4, 5-b]pyrazin-2-ol;
(R)-6-bromo-1-(1-hydroxybutan-2-yl)-1H-imidazo[4, 5-b]pyrazin-2-ol;
(R)-6-bromo-1-(1-morpholinobutan-2-yl)-1H-imidazo[4,5-b]pyrazin-2-ol;
(R)-6-bromo-1-(1-morpholinopropan-2-yl)-1H-imidazo[4,5-b]pyrazin-2-ol;
(R)-6-bromo-1-(1-phenylethyl)-1H-imidazo[4, 5-b]pyrazin-2-ol;
(R)-6-bromo-1-sec-butyl-1H-imidazo[4,5-b]pyrazin-2-ol;
(S)-(2-hydroxy-1-(1-phenylethyl)-1H-imidazo[4,5-b]pyrazin-6-yl)(4-methylpiperazin-1-yl)methanone;
(S)-(2-hydroxy-1-(1-phenylethyl)-1H-imidazo[4,5-b]pyrazin-6-yl)(morpholino)methanone;
(S)-(2-hydroxy-1-(1-phenylethyl)-1H-imidazo[4,5-b]pyrazin-6-yl)(piperidin-1-yl)methanone;
(S)-1-(1-phenylethyl)-1H-imidazo[4,5-b]pyrazin-2-ol;
(S)-1-(1-phenylethyl)-1H-imidazo[4,5-b]quinoxalin-2-ol;
(S)-1-(1-phenylethyl)-6-(piperidin-1-ylmethyl)-1H-imidazo[4,5-b]pyrazin-2-ol;
(S)-1-(1-phenylethyl)-6-propyl-1H-imidazo[4,5-b]pyrazin-2-ol;
(S)-1-(1-phenylethyl)-6-vinyl-1H-imidazo[4,5-b]pyrazin-2-ol;
(S)-1-(2-hydroxy-1-(1-phenylethyl)-1H-imidazo[4,5-b]pyrazin-6-yl)ethanone;
(S)-2-hydroxy-1-(1-phenylethyl)-1H-imidazo[4,5-b]pyrazine-6-carbonitrile;
(S)-2-hydroxy-1-(1-phenylethyl)-1H-imidazo[4, 5-b]pyrazine-6-carboxamide;
(S)-2-hydroxy-1-(1-phenylethyl)-1H-imidazo[4,5-b]pyrazine-6-carboxylic acid;
(S)-2-hydroxy-N,N-dimethyl-1-(1-phenylethyl)-1H-imidazo[4, 5-b]pyrazine-6-carboxamide;
(S)-2-hydroxy-N-methyl-1-(1-phenylethyl)-1H-imidazo[4,5-b]pyrazine-6-carboxamide;
(S)-6-((4-methyl piperazin-1-yl)methyl)-1-(1-phenylethyl)-1H-imidazo[4, 5-b]pyrazin-2-ol;
(S)-6-((dimethylamino)methyl)-1-(1-phenylethyl)-1H-imidazo[4,5-b]pyrazin-2-ol;
(S)-6-(2-hydroxypropan-2-yl)-1-(1-phenylethyl)-1H-imidazo[4,5-b]pyrazin-2-ol;
(S)-6-(2-methylprop-1-enyl)-1-(1-phenylethyl)-1H-imidazo[4,5-b]pyrazin-2-ol;
(S)-6-(methylsulfonyl)-1-(1-phenylethyl)-1H-imidazo[4,5-b]pyrazin-2-ol;
(S)-6-(methylthio)-1-(1-phenylethyl)-1H-imidazo[4, 5-b]pyrazin-2-ol;
(S)-6-(morpholinomethyl)-1-(1-phenylethyl)-1H-imidazo[4,5-b]pyrazin-2-ol;
(S)-6-bromo-1-(1-hydroxybutan-2-yl)-1H-imidazo[4, 5-b]pyrazin-2-ol;
(S)-6-bromo-1-(1-morpholinobutan-2-yl)-1H-imidazo[4,5-b]pyrazin-2-ol;
(S)-6-bromo-1-(1-morpholinopropan-2-yl)-1H-imidazo[4,5-b]pyrazin-2-ol;
(S)-6-bromo-1-(1-phenylethyl)-1H-imidazo[4,5-b]pyrazin-2-ol;
(S)-6-bromo-1-sec-butyl-1H-imidazo[4,5-b]pyrazin-2-ol;
(S)-6-cyclohexenyl-1-(1-phenylethyl)-1H-imidazo[4,5-b]pyrazin-2-ol;
(S)-6-cyclohexyl-1-(1-phenylethyl)-1H-imidazo[4, 5-b]pyrazin-2-ol;
(S)-6-ethoxy-1-(1-phenylethyl)-1H-imidazo[4, 5-b]pyrazin-2-ol;
(S)-6-ethyl-1-(1-phenylethyl)-1H-imidazo[4, 5-b]pyrazin-2-ol;
(S)-6-hexyl-1-(1-phenylethyl)-1H-imidazo[4,5-b]pyrazin-2-ol;
(S)-6-isobutyl-1-(1-phenylethyl)-1H-imidazo[4,5-b]pyrazin-2-ol;
(S)-6-methoxy-1-(1-phenylethyl)-1H-imidazo[4,5-b]pyrazin-2-ol;
(S)-methyl 2-hydroxy-1-(1-phenylethyl)-1H-imidazo[4,5-b]pyrazine-6-carboxylate;
(S)—N, N-diethyl-2-hydroxy-1-(1-phenylethyl)-1H-imidazo[4, 5-b]pyrazine-6-carboxamide;
(S)—N-benzyl-2-hydroxy-1-(1-phenylethyl)-1H-imidazo[4,5-b]pyrazine-6-carboxamide;
(S,E)-1-(1-phenylethyl)-6-(prop-1-enyl)-1H-imidazo[4,5-b]pyrazin-2-ol;
(S,Z)-1-(1-phenylethyl)-6-(prop-1-enyl)-1H-imidazo[4,5-b]pyrazin-2-ol;
(S,Z)-6-(hex-2-enyl)-1-(1-phenylethyl)-1H-imidazo[4, 5-b]pyrazin-2-ol;
(Z)-1-(pentan-3-yl)-6-(prop-1-enyl)-1H-imidazo[4,5-b]pyrazin-2-ol;
(Z)-1-cyclohexyl-6-(prop-1-enyl)-1H-imidazo[4,5-b]pyrazin-2-ol;
(Z)-1-cyclopropyl-6-(prop-1-enyl)-1H-imidazo[4,5-b]pyrazin-2-ol;
(Z)-1-isopropyl-6-(prop-1-enyl)-1H-imidazo[4,5-b]pyrazin-2-ol;
(Z)-6-(prop-1-enyl)-1-(tetrahydro-2H-pyran-4-yl)-1H-imidazo[4, 5-b]pyrazin-2-ol;
1-(1-aminobutan-2-yl)-6-bromo-1H-imidazo[4,5-b]pyrazin-2-ol;
1-(1-morpholinobutan-2-yl)-1H-imidazo[4,5-b]pyrazin-2-ol;
1-(2-hydroxy-1-(pentan-3-yl)-1H-imidazo[4, 5-b]pyrazin-5-yl)ethanone;
1-(pentan-3-yl)-1H-imidazo[4,5-b]pyrazin-2-ol;
1-(pentan-3-yl)-1H-imidazo[4,5-b]pyrazine-2,6-diol;
1-(pentan-3-yl)-1H-imidazo[4,5-b]quinoxalin-2-ol;
1-(pentan-3-yl)-5-vinyl-1H-imidazo[4,5-b]pyrazin-2-ol;
1-(pentan-3-yl)-6-(prop-1-ynyl)-1H-imidazo[4,5-b]pyrazin-2-ol;
1-(pentan-3-yl)-6-(trifluoromethyl)-1H-imidazo[4, 5-b]pyrazin-2-ol;
1-benzyl-6-(methylthio)-1H-imidazo[4, 5-b]pyrazin-2-ol;
1-benzyl-6-bromo-1H-imidazo[4,5-b]pyrazin-2-ol;
1-cyclohexyl-6-(methylthio)-1H-imidazo[4, 5-b]pyrazin-2-ol;
1-cyclopropyl-6-(methylthio)-1H-imidazo[4, 5-b]pyrazin-2-ol;
1-isopropyl-6-(methylthio)-1H-imidazo[4,5-b]pyrazin-2-ol;
2-(6-bromo-2-hydroxy-1H-imidazo[4, 5-b]pyrazin-1-yl)-1-morpholinobutan-1-one;
2-(6-bromo-2-hydroxy-1H-imidazo[4,5-b]pyrazin-1-yl)butanoic acid;
2-(6-bromo-2-hydroxy-1H-imidazo[4, 5-b]pyrazin-1-yl)propane-1,3-diol;
2-hydroxy-1-(pentan-3-yl)-1H-imidazo[4,5-b]pyrazine-5-carboxylic acid;
2-hydroxy-1-(pentan-3-yl)-1H-imidazo[4, 5-b]pyrazine-6-carbonitrile;
2-hydroxy-N, N-dimethyl-1-(pentan-3-yl)-1H-imidazo[4,5-b]pyrazine-5-carboxamide;
2-hydroxy-N-methyl-1-(pentan-3-yl)-1H-imidazo[4,5-b]pyrazine-5-carboxamide;
5-(methylthio)-1-(pentan-3-yl)-1H-imidazo[4,5-b]pyrazin-2-ol;
5-bromo-1-(pentan-3-yl)-1H-imidazo[4,5-b]pyrazin-2-ol;
5-ethyl-1-(pentan-3-yl)-1H-imidazo[4,5-b]pyrazin-2-ol;
6-(methylsulfinyl)-1-((S)-1-phenylethyl)-1H-imidazo[4, 5-b]pyrazin-2-ol;
6-(methylthio)-1-(pentan-3-yl)-1H-imidazo[4,5-b]pyrazin-2-ol;
6-(methylthio)-1-(tetrahydro-2H-pyran-4-yl)-1H-imidazo[4,5-b]pyrazin-2-ol;
6-bromo-1-(1-(4-(methylsulfonyl)piperazin-1-yl)butan-2-yl)-1H-imidazo[4,5-b]pyrazin-2-ol;
6-bromo-1-(1-(4-methylpiperazin-1-yl)butan-2-yl)-1H-imidazo[4, 5-b]pyrazin-2-ol;
6-bromo-1-(1-(dimethylamino)butan-2-yl)-1H-imidazo[4,5-b]pyrazin-2-ol;
6-bromo-1-(1-(methylamino)butan-2-yl)-1H-imidazo[4,5-b]pyrazin-2-ol;
6-bromo-1-(1-methoxybutan-2-yl)-1H-imidazo[4, 5-b]pyrazin-2-ol;
6-bromo-1-(2-methyl-1-morpholinopropan-2-yl)-1H-imidazo[4,5-b]pyrazin-2-ol;
6-bromo-1-(2-morpholinoethyl)-1H-imidazo[4,5-b]pyrazin-2-ol;
6-bromo-1-(pentan-3-yl)-1H-imidazo[4,5-b]pyrazin-2-ol;
6-bromo-1-(tetrahydro-2H-pyran-4-yl)-1H-imidazo[4,5-b]pyrazin-2-ol;
6-bromo-1-cyclohexyl-1H-imidazo[4,5-b]pyrazin-2-ol;
6-bromo-1-cyclopropyl-1H-imidazo[4,5-b]pyrazin-2-ol;
6-bromo-1-isopropyl-1H-imidazo[4,5-b]pyrazin-2-ol;
6-bromo-1-tert-butyl-1H-imidazo[4,5-b]pyrazin-2-ol;
6-cyclopropyl-1-(pentan-3-yl)-1H-imidazo[4,5-b]pyrazin-2-ol;
6-ethynyl-1-(pentan-3-yl)-1H-imidazo[4,5-b]pyrazin-2-ol;
6-m ethoxy-1-(pentan-3-yl)-1H-imidazo[4, 5-b]pyrazin-2-ol;
6-methyl-1-(pentan-3-yl)-1H-imidazo[4, 5-b]pyrazin-2-ol;
methyl 2-hydroxy-1-(pentan-3-yl)-1H-imidazo[4,5-b]pyrazine-5-carboxylate;
methyl
4-(2-(6-bromo-2-hydroxy-1H-imidazo[4,5-b]pyrazin-1-yl)butyl)piperazine-1-carboxylate;
1-(ethylpropyl)-6-(1-methylpyrazol-4-yl)imidazo[4,5-b]pyrazin-2-ol;
6-bromo-1-(propylbutyl)imidazo[4, 5-b]pyrazin-2-ol;
1-[(1R)-3-methyl-1-(morpholin-4-ylmethyl)butyl]-6-bromoimidazo[4,5-b]pyrazin-2-ol;
1-(ethylpropyl)-6-vinylimidazo[4,5-b]pyrazin-2-ol;
1-(ethyl propyl)-6-(1-methylvinyl)imidazo[4,5-b]pyrazin-2-ol;
1-(ethylpropyl)-6-(methylethyl)imidazo[4,5-b]pyrazin-2-ol;
6-chloro-1-(ethylpropyl)imidazo[4,5-b]pyrazin-2-ol; and
6-(dimethylamino)-1-(ethylpropyl)imidazo[4,5-b]pyrazin-2-ol,

or a pharmaceutically acceptable salt thereof.

In some embodiments, the compound of Formula B is chosen from the following tautomers of compounds of Formula A:
(R)-6-bromo-1-(1-morpholinopropan-2-yl)-1H-imidazo[4,5-b]pyrazin-2(3H)-one;
6-ethynyl-1-(pentan-3-yl)-1H-imidazo[4,5-b]pyrazin-2(3H)-one;
6-methoxy-1-(pentan-3-yl)-1H-imidazo[4,5-b]pyrazin-2(3H)-one;
6-bromo-1-(2-methyl-1-morpholinopropan-2-yl)-1H-imidazo[4,5-b]pyrazin-2(3H)-one;
1-(pentan-3-yl)-6-(1H-1,2,3-triazol-4-yl)-1H-imidazo[4,5-b]pyrazin-2(3H)-one;
1-(pentan-3-yl)-6-(trifluoromethyl)-1H-imidazo[4, 5-b]pyrazin-2(3H)-one;
(R)-6-ethynyl-1-(1-morpholinobutan-2-yl)-1H-imidazo[4, 5-b]pyrazin-2(3H)-one;
6-((2-hydroxy-1-(pentan-3-yl)-1H-imidazo[4,5-b]pyrazin-6-yl)ethynyl)-1-(pentan-3-yl)-1H-imidazo[4,5-b]pyrazin-2(3H)-one;
6-(dimethylamino)-1-(pentan-3-yl)-1H-imidazo[4,5-b]pyrazin-2(3H)-one;
6-ethyl-1-(pentan-3-yl)-1H-imidazo[4,5-b]pyrazin-2(3H)-one;
(E)-1-(pentan-3-yl)-6-(prop-1-enyl)-1H-imidazo[4,5-b]pyrazin-2(3H)-one;
(E)-1-cyclohexyl-6-(prop-1-enyl)-1H-imidazo[4,5-b]pyrazin-2(3H)-one;
(E)-1-cyclopropyl-6-(prop-1-enyl)-1H-imidazo[4,5-b]pyrazin-2(3H)-one;
(E)-1-isopropyl-6-(prop-1-enyl)-1H-imidazo[4,5-b]pyrazin-2(3H)-one;
(E)-6-(prop-1-enyl)-1-(tetrahydro-2H-pyran-4-yl)-1H-imidazo[4,5-b]pyrazin-2(3H)-one;
(R)-6-(methylthio)-1-(1-phenylethyl)-1H-imidazo[4,5-b]pyrazin-2(3H)-one;
(R)-6-bromo-1-(1-hydroxybutan-2-yl)-1H-imidazo[4,5-b]pyrazin-2(3H)-one;
(R)-6-bromo-1-(1-morpholinobutan-2-yl)-1H-imidazo[4,5-b]pyrazin-2(3H)-one;
(R)-6-bromo-1-(1-morpholinopropan-2-yl)-1H-imidazo[4,5-b]pyrazin-2(3H)-one;
(R)-6-bromo-1-(1-phenylethyl)-1H-imidazo[4,5-b]pyrazin-2(3H)-one;
(R)-6-bromo-1-sec-butyl-1H-imidazo[4,5-b]pyrazin-2(3H)-one;
(S)-1-(1-phenylethyl)-1H-imidazo[4,5-b]pyrazin-2(3H)-one;
(S)-1-(1-phenylethyl)-1H-imidazo[4,5-b]quinoxalin-2(3H)-one;
(S)-1-(1-phenylethyl)-6-(piperidin-1-ylmethyl)-1H-imidazo[4,5-b]pyrazin-2(3H)-one;
(S)-1-(1-phenylethyl)-6-(piperidine-1-carbonyl)-1H-imidazo[4, 5-b]pyrazin-2(3H)-one;
(S)-1-(1-phenylethyl)-6-propyl-1H-imidazo[4,5-b]pyrazin-2(3H)-one;
(S)-1-(1-phenylethyl)-6-vinyl-1H-imidazo[4,5-b]pyrazin-2(3H)-one;
(S)-2-oxo-3-(1-phenylethyl)-2,3-dihydro-1H-imidazo[4,5-b]pyrazine-5-carbonitrile;
(S)-2-oxo-3-(1-phenylethyl)-2,3-dihydro-1H-imidazo[4,5-b]pyrazine-5-carboxamide;
(S)-2-oxo-3-(1-phenylethyl)-2, 3-dihydro-1H-imidazo[4, 5-b]pyrazine-5-carboxylic acid;
(S)-6-((4-methyl piperazin-1-yl)methyl)-1-(1-phenylethyl)-1H-imidazo[4,5-b]pyrazin-2(3H)-one;
(S)-6-((dimethylamino)methyl)-1-(1-phenylethyl)-1H-imidazo[4,5-b]pyrazin-2(3H)-one;
(S)-6-(2-hydroxypropan-2-yl)-1-(1-phenylethyl)-1H-imidazo[4,5-b]pyrazin-2(3H)-one;
(S)-6-(2-methyl prop-1-enyl)-1-(1-phenylethyl)-1H-imidazo[4,5-b]pyrazin-2(3H)-one;
(S)-6-(4-methyl piperazine-1-carbonyl)-1-(1-phenylethyl)-1H-imidazo[4,5-b]pyrazin-2(3H)-one;
(S)-6-(methylsulfonyl)-1-(1-phenylethyl)-1H-imidazo[4,5-b]pyrazin-2(3H)-one;
(S)-6-(methylthio)-1-(1-phenylethyl)-1H-imidazo[4,5-b]pyrazin-2(3H)-one;
(S)-6-(morpholine-4-carbonyl)-1-(1-phenylethyl)-1H-imidazo[4,5-b]pyrazin-2(3H)-one;
(S)-6-(morpholinomethyl)-1-(1-phenylethyl)-1H-imidazo[4,5-b]pyrazin-2(3H)-one;
(S)-6-acetyl-1-(1-phenylethyl)-1H-imidazo[4,5-b]pyrazin-2(3H)-one;
(S)-6-bromo-1-(1-hydroxybutan-2-yl)-1H-imidazo[4,5-b]pyrazin-2(3H)-one;
(S)-6-bromo-1-(1-morpholinobutan-2-yl)-1H-imidazo[4,5-b]pyrazin-2(3H)-one;
(S)-6-bromo-1-(1-morpholinopropan-2-yl)-1H-imidazo[4,5-b]pyrazin-2(3H)-one;
(S)-6-bromo-1-(1-phenylethyl)-1H-imidazo[4,5-b]pyrazin-2(3H)-one;
(S)-6-bromo-1-sec-butyl-1H-imidazo[4,5-b]pyrazin-2(3H)-one;
(S)-6-cyclohexenyl-1-(1-phenylethyl)-1H-imidazo[4,5-b]pyrazin-2(3H)-one;
(S)-6-cyclohexyl-1-(1-phenylethyl)-1H-imidazo[4,5-b]pyrazin-2(3H)-one;
(S)-6-ethoxy-1-(1-phenylethyl)-1H-imidazo[4,5-b]pyrazin-2(3H)-one;
(S)-6-ethyl-1-(1-phenylethyl)-1H-imidazo[4,5-b]pyrazin-2(3H)-one;
(S)-6-hexyl-1-(1-phenylethyl)-1H-imidazo[4,5-b]pyrazin-2(3H)-one;
(S)-6-isobutyl-1-(1-phenylethyl)-1H-imidazo[4,5-b]pyrazin-2(3H)-one;
(S)-6-methoxy-1-(1-phenylethyl)-1H-imidazo[4,5-b]pyrazin-2(3H)-one;
(S)-methyl
2-oxo-3-(1-phenylethyl)-2,3-dihydro-1H-imidazo[4,5-b]pyrazine-5-carboxylate;
(S)—N, N-diethyl-2-oxo-3-(1-phenylethyl)-2,3-dihydro-1H-imidazo[4,5-b]pyrazine-5-carboxamide;
(S)—N, N-dimethyl-2-oxo-3-(1-phenylethyl)-2,3-dihydro-1H-imidazo[4,5-b]pyrazine-5-carboxamide;
(S)—N-benzyl-2-oxo-3-(1-phenylethyl)-2,3-dihydro-1H-imidazo[4,5-b]pyrazine-5-carboxamide;
(S)—N-methyl-2-oxo-3-(1-phenylethyl)-2,3-dihydro-1H-imidazo[4,5-b]pyrazine-5-carboxamide;
(S,E)-1-(1-phenylethyl)-6-(prop-1-enyl)-1H-imidazo[4,5-b]pyrazin-2(3H)-one;
(S,Z)-1-(1-phenylethyl)-6-(prop-1-enyl)-1H-imidazo[4,5-b]pyrazin-2(3H)-one;
(S,Z)-6-(hex-2-enyl)-1-(1-phenylethyl)-1H-imidazo[4,5-b]pyrazin-2(3H)-one;
(Z)-1-(pentan-3-yl)-6-(prop-1-enyl)-1H-imidazo[4,5-b]pyrazin-2(3H)-one;
(Z)-1-cyclohexyl-6-(prop-1-enyl)-1H-imidazo[4,5-b]pyrazin-2(3H)-one;
(Z)-1-cyclopropyl-6-(prop-1-enyl)-1H-imidazo[4,5-b]pyrazin-2(3H)-one;
(Z)-1-isopropyl-6-(prop-1-enyl)-1H-imidazo[4,5-b]pyrazin-2(3H)-one;
(Z)-6-(prop-1-enyl)-1-(tetrahydro-2H-pyran-4-yl)-1H-imidazo[4,5-b]pyrazin-2(3H)-one;
1-(1-aminobutan-2-yl)-6-bromo-1H-imidazo[4,5-b]pyrazin-2(3H)-one;
1-(1-morpholinobutan-2-yl)-1H-imidazo[4,5-b]pyrazin-2(3H)-one;
1-(pentan-3-yl)-1H-imidazo[4,5-b]pyrazin-2(3H)-one;
1-(pentan-3-yl)-1H-imidazo[4,5-b]quinoxalin-2(3H)-one;
1-(pentan-3-yl)-5-vinyl-1H-imidazo[4,5-b]pyrazin-2(3H)-one;
1-(pentan-3-yl)-6-(prop-1-ynyl)-1H-imidazo[4,5-b]pyrazin-2(3H)-one;
1-(pentan-3-yl)-6-(trifluoromethyl)-1H-imidazo[4,5-b]pyrazin-2(3H)-one;
1-benzyl-6-(methylthio)-1H-imidazo[4,5-b]pyrazin-2(3H)-one;
1-benzyl-6-bromo-1H-imidazo[4,5-b]pyrazin-2(3H)-one;
1-cyclohexyl-6-(methylthio)-1H-imidazo[4,5-b]pyrazin-2(3H)-one;
1-cyclopropyl-6-(methylthio)-1H-imidazo[4,5-b]pyrazin-2(3H)-one;
1-isopropyl-6-(methylthio)-1H-imidazo[4,5-b]pyrazin-2(3H)-one;
2-(6-bromo-2-oxo-2,3-dihydro-1H-imidazo[4,5-b]pyrazin-1-yl)butanoic acid;
2-oxo-1-(pentan-3-yl)-2,3-dihydro-1H-imidazo[4,5-b]pyrazine-5-carboxylic acid;
2-oxo-3-(pentan-3-yl)-2,3-dihydro-1H-imidazo[4, 5-b]pyrazine-5-carbonitrile;
5-(methylthio)-1-(pentan-3-yl)-1H-imidazo[4,5-b]pyrazin-2(3H)-one;
5-acetyl-1-(pentan-3-yl)-1H-imidazo[4,5-b]pyrazin-2(3H)-one;
5-bromo-1-(pentan-3-yl)-1H-imidazo[4,5-b]pyrazin-2(3H)-one;
5-ethyl-1-(pentan-3-yl)-1H-imidazo[4,5-b]pyrazin-2(3H)-one;
6-(methylsulfinyl)-1-((S)-1-phenylethyl)-1H-imidazo[4, 5-b]pyrazin-2(3H)-one;
6-(methylthio)-1-(pentan-3-yl)-1H-imidazo[4,5-b]pyrazin-2(3H)-one;
6-(methylthio)-1-(tetrahydro-2H-pyran-4-yl)-1H-imidazo[4,5-b]pyrazin-2(3H)-one;
6-bromo-1-(1-(4-(methylsulfonyl)piperazin-1-yl)butan-2-yl)-1H-imidazo[4,5-b]pyrazin-2(3H)-one;
6-bromo-1-(1-(4-methylpiperazin-1-yl)butan-2-yl)-1H-imidazo[4,5-b]pyrazin-2(3H)-one;
6-bromo-1-(1-(dimethylamino)butan-2-yl)-1H-imidazo[4,5-b]pyrazin-2(3H)-one;
6-bromo-1-(1-(methylamino)butan-2-yl)-1H-imidazo[4,5-b]pyrazin-2(3H)-one;
6-bromo-1-(1,3-dihydroxypropan-2-yl)-1H-imidazo[4,5-b]pyrazin-2(3H)-one;
6-bromo-1-(1-methoxybutan-2-yl)-1H-imidazo[4,5-b]pyrazin-2(3H)-one;
6-bromo-1-(1-morpholino-1-oxobutan-2-yl)-1H-imidazo[4,5-b]pyrazin-2(3H)-one;
6-bromo-1-(2-methyl-1-morpholinopropan-2-yl)-1H-imidazo[4,5-b]pyrazin-2(3H)-one;
6-bromo-1-(2-morpholinoethyl)-1H-imidazo[4,5-b]pyrazin-2(3H)-one;
6-bromo-1-(pentan-3-yl)-1H-imidazo[4,5-b]pyrazin-2(3H)-one;
6-bromo-1-(tetrahydro-2H-pyran-4-yl)-1H-imidazo[4,5-b]pyrazin-2(3H)-one;
6-bromo-1-cyclohexyl-1H-imidazo[4,5-b]pyrazin-2(3H)-one;
6-bromo-1-cyclopropyl-1H-imidazo[4,5-b]pyrazin-2(3H)-one;
6-bromo-1-isopropyl-1H-imidazo[4,5-b]pyrazin-2(3H)-one;
6-bromo-1-tert-butyl-1H-imidazo[4,5-b]pyrazin-2(3H)-one;
6-cyclopropyl-1-(pentan-3-yl)-1H-imidazo[4, 5-b]pyrazin-2(3H)-one;
6-ethynyl-1-(pentan-3-yl)-1H-imidazo[4, 5-b]pyrazin-2(3H)-one;
6-hydroxy-1-(pentan-3-yl)-1H-imidazo[4,5-b]pyrazin-2(3H)-one;
6-methoxy-1-(pentan-3-yl)-1H-imidazo[4,5-b]pyrazin-2(3H)-one;
6-methyl-1-(pentan-3-yl)-1H-imidazo[4,5-b]pyrazin-2(3H)-one;
methyl
2-oxo-1-(pentan-3-yl)-2,3-dihydro-1H-imidazo[4, 5-b]pyrazine-5-carboxylate;
methyl
4-(2-(6-bromo-2-oxo-2,3-dihydro-1H-imidazo[4,5-b]pyrazin-1-yl)butyl)piperazine-1-carboxylate;
N,N-dimethyl-2-oxo-1-(pentan-3-yl)-2,3-dihydro-1H-imidazo[4, 5-b]pyrazine-5-carb
oxamide;
N-methyl-2-oxo-1-(pentan-3-yl)-2,3-dihydro-1H-imidazo[4,5-b]pyrazine-5-carboxamide;
6-(1-methyl-1H-pyrazol-4-yl)-1-(pentan-3-yl)-1H-imidazo[4,5-b]pyrazin-2(3H)-one;
6-bromo-1-(heptan-4-yl)-1H-imidazo[4,5-b]pyrazin-2(3H)-one;
(R)-6-bromo-1-(4-methyl-1-morpholinopentan-2-yl)-1H-imidazo[4,5-b]pyrazin-2(3H)-one;
1-(pentan-3-yl)-6-vinyl-1H-imidazo[4,5-b]pyrazin-2(3H)-one;
1-(pentan-3-yl)-6-(prop-1-en-2-yl)-1H-imidazo[4,5-b]pyrazin-2(3H)-one;
6-isopropyl-1-(pentan-3-yl)-1H-imidazo[4,5-b]pyrazin-2(3H)-one;
6-chloro-1-(pentan-3-yl)-1H-imidazo[4,5-b]pyrazin-2(3H)-one; and
6-(dimethylamino)-1-(pentan-3-yl)-1H-imidazo[4,5-b]pyrazin-2(3H)-one,

or a pharmaceutically acceptable salt thereof.

In some embodiments, the compound of Formula A is 6-bromo-1-(ethylpropyl)imidazo[4,5-b]pyrazin-2-ol (Compound A) or a pharmaceutically acceptable salt thereof. In some embodiments, the compound of formula A is 1-(ethylpropyl)-6-ethynylimidazo[4,5-b]pyrazin-2-ol (Compound C) or a pharmaceutically acceptable salt thereof.

The compounds of Formula A can be named and numbered (e.g., using NamExpert™ available from Cheminnovation or the automatic naming feature of ChemDraw Ultra version 10.0 from Cambridge Soft Corporation) as described below. For example, the compound:

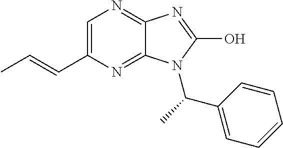 [SMILES: C/C=C/c1cnc2nc(O)n([C@@H](C)c3ccccc3)c2n1]

i.e., the compound according to Formula A where R₁ is (E)-propen-1yl, R₂ is (S)-sec-phenethyl, and R₄ is H, can be named (S,E)-1-(1-phenylethyl)-6-(prop-1-enyl)-1H-imidazo[4,5-b]pyrazin-2-ol.

Likewise the compound:

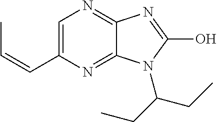 [SMILES: C/C=C\c1cnc2nc(O)n(C(CC)CC)c2n1]

i.e., the compound according to Formula A where R₁ is (Z)-propen-1-yl, R₂ is 3-pentyl, and R₄ is H, can be named (Z)-1-(pentan-3-yl)-6-(prop-1-enyl)-1H-imidazo[4,5-b]pyrazin-2-ol.

Similarly, the compounds of Formula B can be named and numbered (e.g., using NamExpert™ available from Cheminnovation or the automatic naming feature of ChemDraw Ultra version 10.0 from Cambridge Soft Corporation) as described below. For example, the compound:

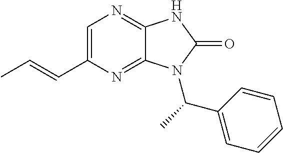 [SMILES: C/C=C/c1cnc2[nH]c(=O)n([C@@H](C)c3ccccc3)c2n1]

i.e., the compound according to Formula B where R₁ is (E)-propen-1-yl, R₂ is (S)-sec-phenethyl, and R₄ is H, can be named (S,E)-1-(1-phenylethyl)-6-(prop-1-enyl)-1H-imidazo[4,5-b]pyrazin-2(3H)-one.

Likewise the compound:

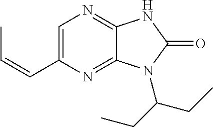 [SMILES: C/C=C\c1cnc2[nH]c(=O)n(C(CC)CC)c2n1]

i.e., the compound according to Formula B where R₁ is (Z)-propen-1-yl, R₂ is 3-pentyl, and R₄ is H, can be named (Z)-1-(pentan-3-yl)-6-(prop-1-enyl)-1H-imidazo[4,5-b]pyrazin-2(3H)-one.

In some embodiments, a skeletal muscle troponin activator is a compound of Formula I:

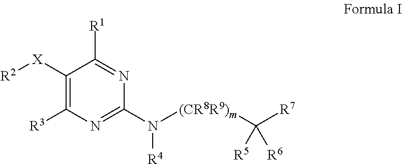

or a pharmaceutically acceptable salt thereof, wherein:

R¹ is selected from hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl and 5-10 membered heteroaryl;

R² is selected from C₃₋₈ cycloalkyl, C₃₋₈ cycloalkenyl, 3-8 membered heterocycloalkyl, 3-8 membered heterocycloalkenyl, C₆₋₁₀ aryl, 5-10 membered heteroaryl and NR^{b}R^{c}, wherein each of the C₃₋₈ cycloalkyl, C₃₋₈ cycloalkenyl, 3-8 membered heterocycloalkyl, 3-8 membered heterocycloalkenyl, C₆₋₁₀ aryl and 5-10 membered heteroaryl groups is optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙC₆₋₁₀ aryl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙC₆₋₁₀ aryl and (CH₂)ₙ5-10 membered heteroaryl groups is optionally substituted with 1, 2, 3, 4 or 5 R^{f} substituents;

R³ is selected from hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl and 5-10 membered heteroaryl;

R⁴ is selected from hydrogen, C₁₋₆ alkyl, C₁₋₆ haloalkyl, C(O)R^{a}, C(O)OR^{a}, C(O)NR^{b}R^{c} and SO₂R^{a};

R⁵ and R⁶ are each independently selected from hydrogen, halogen, C₁₋₆ alkyl and C₁₋₆ haloalkyl;

or alternatively, R⁵ and R⁶ together with the carbon atom to which they are bound form a group selected from C₃₋₈ cycloalkyl, C₃₋₈ cycloalkenyl, 3-8 membered heterocycloalkyl and 3-8 membered heterocycloalkenyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl;

R⁷ is selected from C₃₋₈ cycloalkyl, C₃₋₈ cycloalkenyl, 3-8 membered heterocycloalkyl, 3-8 membered heterocycloalkenyl, C₆₋₁₀ aryl and 5-10 membered heteroaryl, each optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents;

R⁸ and R⁹, at each occurrence, are each independently selected from hydrogen, halogen and C₁₋₆ alkyl;

X is selected from a bond, —(CH₂)ₚ—, —(CH₂)ₚC(O)(CH₂)_{q}—, —(CH₂)ₚO(CH₂)_{q}—, —(CH₂)ₚS(CH₂)_{q}—, —(CH₂)ₚNR^{d}(CH₂)_{q}—, —(CH₂)ₚC(O)O(CH₂)_{q}—, —(CH₂)ₚOC(O)(CH₂)_{q}—, —(CH₂)ₚNR^{d}C(O)(CH₂)_{q}—, —(CH₂)ₚC(O)NR^{d}(CH₂)_{q}—, —(CH₂)ₚNR^{d}C(O)NR^{d}(CH₂)_{q}—, —(CH₂)ₚNR^{d}SO₂(CH₂)_{q}—, and —(CH₂)ₚSO₂NR^{d}(CH₂)_{q}—;

or alternatively, X, R² and R³, together with the carbon atoms to which they are bound, form a 5-6 membered ring optionally containing one or more heteroatoms selected from oxygen nitrogen and sulfur, and optionally containing one or more double bonds, and optionally substituted with 1, 2, 3, 4 or 5 R^{f} substituents;

R^{a}, at each occurrence, is independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl, wherein each of the C₁₋₆ alkyl, O₂-6 alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents;

R^{b} and R^{c}, at each occurrence, are each independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, 5-10 membered heteroaryl, C(O)R^{g}, C(O)OR^{g}, C(O)NRⁱR^{j} and SO₂R^{g}, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents;

R^{d}, at each occurrence, is independently selected from hydrogen and C₁₋₆ alkyl;

R^{e}, at each occurrence, is independently selected from hydrogen, CN, OH, C₁₋₆ alkoxy, C₁₋₆ alkyl and C₁₋₆ haloalkyl;

R^{f}, at each occurrence, is independently selected from halogen, CN, OR^{h}, OC(O)R^{h}, OC(O)OR^{h}, OC(O)NRⁱR^{j}, NRⁱR^{j}, NR^{d}C(O)R^{h}, NR^{d}C(O)OR^{h}, NR^{d}C(O)NRⁱR^{j}, NR^{d}C(O)C(O)NRⁱR^{j}, NR^{d}C(S)R^{h}, NR^{d}C(S)OR^{h}, NR^{d}C(S)NRⁱR^{j}, NR^{d}C(NR^{e})NRⁱR^{j}, NR^{d}S(O)R^{h}, NR^{d}SO₂R^{h}, NR^{d}SO₂NRⁱR^{j}, C(O)R^{h}, C(O)OR^{h}, C(O)NRⁱR^{j}, C(S)R^{h}, C(S)OR^{h}, C(S)NRⁱR^{j}, C(NR^{e})NRⁱR^{j}, SR^{h}, S(O)R^{h}, SO₂R^{h}, SO₂NRⁱR^{j}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{k} substituents;

or two R^{f} substituents bound to a single carbon atom, together with the carbon atom to which they are both bound, form a group selected from carbonyl, C₃₋₈ cycloalkyl and 3-8 membered heterocycloalkyl;

R^{g}, at each occurrence, is independently selected from C₁₋₆ alkyl, C₁₋₆ haloalkyl, phenyl, naphthyl, and C₇₋₁₁ aralkyl, each optionally substituted with 1, 2, 3, 4 or 5 substituents selected from halogen, CN, OH, C₁₋₆ alkoxy, C₁₋₆ alkyl and C₁₋₆ haloalkyl;

R^{h}, at each occurrence, is independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{k} substituents;

Rⁱ and R^{j}, at each occurrence, are each independently selected from hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, 5-10 membered heteroaryl, C(O)R^{g}, and C(O)OR^{g}, wherein each of the C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 substituents selected from halogen, CN, OH, C₁₋₆ alkoxy, C₁₋₆ alkyl and C₁₋₆ haloalkyl;

R^{k}, at each occurrence, is independently selected from halogen, CN, OH, C₁₋₆ alkoxy, NH₂, NH(C₁₋₆ alkyl), N(C₁₋₆ alkyl)₂, NHC(O)C₁₋₆ alkyl, NHC(O)C₇₋₁₁ aralkyl, NHC(O)OC₁₋₆ alkyl, NHC(O)OC₇₋₁₁ aralkyl, OC(O)C₁₋₆ alkyl, OC(O)C₇₋₁₁ aralkyl, OC(O)OC₁₋₆ alkyl, OC(O)OC₇₋₁₁ aralkyl, C(O)C₁₋₆ alkyl, C(O)C₇₋₁₁ aralkyl, C(O)OC₁₋₆ alkyl, C(O)OC₇₋₁₁ aralkyl, C₁₋₆ alkyl, C₁₋₆ haloalkyl, C₂₋₆ alkenyl, and C₂₋₆ alkynyl, wherein each C₁₋₆ alkyl, C₂₋₆ alkenyl, C₂₋₆ alkynyl, and C₇₋₁₁ aralkyl substituent is optionally substituted with 1, 2 or 3 substituents selected from OH, C₁₋₆ alkoxy, NH₂, NH(C₁₋₆ alkyl), N(C₁₋₆ alkyl)₂, NHC(O)C₁₋₆ alkyl, NHC(O)C₇₋₁₁ aralkyl, NHC(O)OC₁₋₆ alkyl, and NHC(O)OC₇₋₁₁ aralkyl;

or two R^{k} substituents bound to a single carbon atom, together with the carbon atom to which they are both bound, form a carbonyl group;

m is 0, 1 or 2;

n, at each occurrence, independently is 0, 1 or 2;

p is 0, 1 or 2; and

q is 0, 1 or 2.

In some embodiments of compounds of Formula I, m is 0, i.e., a compound of Formula II, or a pharmaceutically acceptable salt thereof:

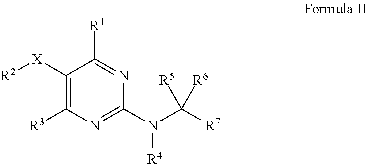

wherein R¹, R², R³, R⁴, R⁵, R⁶, R⁷ and X are as defined herein.

In some embodiments of compounds of Formula I, m is 1, i.e., a compound of Formula III, or a pharmaceutically acceptable salt thereof:

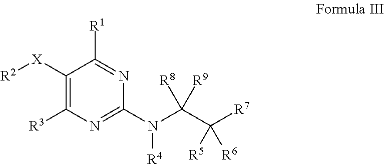

wherein R¹, R², R³, R⁴, R⁵, R⁶, R⁷, R⁸, R⁹ and X are as defined herein.

In some embodiments, one of R⁵ and R⁶ is hydrogen and the other is C₁₋₆ alkyl.

In some embodiments, R⁵ and R⁶ are each independently C₁₋₆ alkyl.

In some embodiments, R⁵ and R⁶ are each methyl.

In some embodiments, the compounds are of Formula IV(a) or IV(b), or a pharmaceutically acceptable salt thereof:

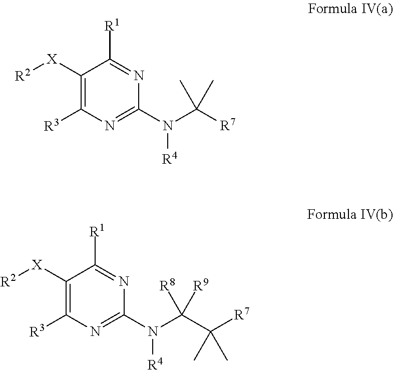

wherein R¹, R², R³, R⁴, R⁷, R⁸, R⁹ and X are as defined herein.

In some embodiments, R⁵ and R⁶ together with the carbon atom to which they are bound form C₃₋₈ cycloalkyl, C₃₋₈ cycloalkenyl, 3-8 membered heterocycloalkyl or 3-8 membered heterocycloalkenyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments, R⁵ and R⁶, together with the carbon to which they are bound, form C₃₋₆ cycloalkyl optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments, R⁵ and R⁶, together with the carbon to which they are bound, form cyclopropyl, cyclobutyl, cyclopentyl or cyclohexyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments, R⁵ and R⁶, together with the carbon to which they are bound, form cyclobutyl optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments, R⁵ and R⁶, together with the carbon to which they are bound, form cyclobutyl substituted with one substituent selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl, wherein the substituent and R⁷ are in a trans configuration with respect to one another on the cyclobutyl ring.

In some embodiments, R⁵ and R⁶, together with the carbon to which they are bound, form cyclobutyl substituted with one substituent selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl, wherein the substituent and R⁷ are in a cis configuration with respect to one another on the cyclobutyl ring.

In some embodiments, the compounds are of Formula V(a) or V(b), or a pharmaceutically acceptable salt thereof:

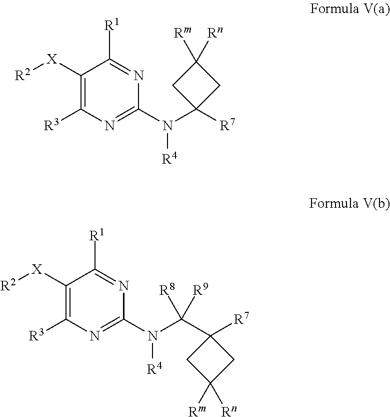

wherein R^{m} and Rⁿ are each independently selected from hydrogen, halogen and C₁₋₆ alkyl, and R¹, R², R³, R⁴, R⁷, R⁸, R⁹ and X are as defined herein.

In some embodiments, R^{m} and Rⁿ are each hydrogen.

In some embodiments, R^{m} and Rⁿ are each halogen.

In some embodiments, R^{m} and Rⁿ are each fluorine.

In some embodiments, one of R^{m} and Rⁿ is hydrogen and the other is halogen. In some embodiments of such compounds, the halogen and R⁷ are in a trans configuration with respect to one another on the cyclobutyl ring. In some embodiments of such compounds, the halogen and R⁷ are in a cis configuration with respect to one another on the cyclobutyl ring.

In some embodiments, one of R^{m} and Rⁿ is hydrogen and the other is fluorine. In some embodiments of such compounds, the fluorine and R⁷ are in a trans configuration with respect to one another on the cyclobutyl ring. In some embodiments of such compounds, the fluorine and R⁷ are in a cis configuration with respect to one another on the cyclobutyl ring.

In some embodiments, R⁵ and R⁶, together with the carbon atom to which they are bound, form 3-6 membered heterocycloalkyl, each of which is optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments, R⁵ and R⁶, together with the carbon atom to which they are bound, form aziridine, azetidine, pyrrolidine, oxirane, oxetane or tetrahydrofuran, each of which is optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments, R⁵ and R⁶ are each independently C₁₋₆ alkyl, or R⁵ and R⁶ together with the carbon atom to which they are bound form C₃₋₈ cycloalkyl, C₃₋₈ cycloalkenyl, 3-8 membered heterocycloalkyl or 3-8 membered heterocycloalkenyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments, R⁵ and R⁶ are each methyl, or R⁵ and R⁶ together with the carbon atom to which they are bound form C₃₋₈ cycloalkyl, C₃₋₈ cycloalkenyl, 3-8 membered heterocycloalkyl or 3-8 membered heterocycloalkenyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments, R⁵ and R⁶ are each independently C₁₋₆ alkyl, or R⁵ and R⁶, together with the carbon to which they are bound, form cyclopropyl, cyclobutyl, cyclopentyl or cyclohexyl, each optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments, R⁵ and R⁶ are each methyl, or R⁵ and R⁶, together with the carbon to which they are bound, form cyclobutyl optionally substituted with 1, 2, 3, 4 or 5 substituents selected from halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl and C₁₋₆ haloalkyl.

In some embodiments, R⁷ is selected from C₃₋₈ cycloalkyl, C₃₋₈ cycloalkenyl, 3-8 membered heterocycloalkyl, 3-8 membered heterocycloalkenyl, C₆₋₁₀ aryl and 5-10 membered heteroaryl, each optionally substituted with 1,2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents.

In some embodiments, R⁷ is phenyl optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents.

In some embodiments, the compounds are of Formula VI, or a pharmaceutically acceptable salt thereof:

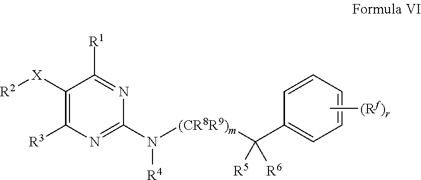

wherein r is 0, 1, 2, 3 or 4, and R¹, R², R³, R⁴, R⁵, R⁶, R⁸, R⁹, R^{f}, X and m are as defined herein.

In some embodiments, the compounds are of Formula VII(a) or VII(b), or a pharmaceutically acceptable salt thereof:

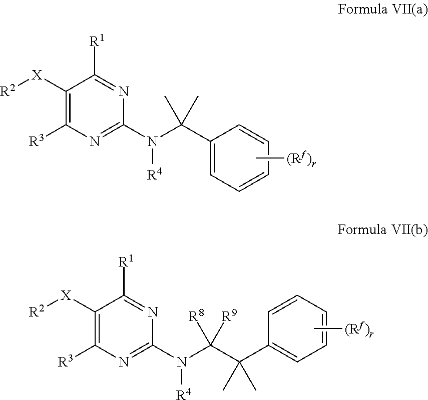

wherein r is 0, 1,2, 3 or 4, and R¹, R², R³, R⁴, R⁸, R⁹, R^{f} and X are as defined herein.

In some embodiments, the compounds are of Formula VIII(a) or VIII(b), or a pharmaceutically acceptable salt thereof:

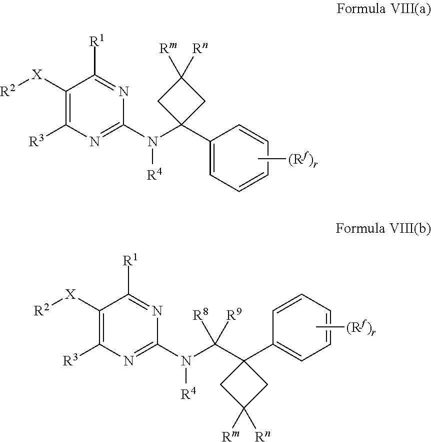

wherein R^{m} and Rⁿ are each independently selected from hydrogen, halogen and C₁₋₆ alkyl; r is 0, 1, 2, 3 or 4; and R¹, R², R³, R⁴, R⁸, R⁹, R^{f} and X are as defined herein.

In some embodiments, R^{m} and Rⁿ are each hydrogen.

In some embodiments, R^{m} and Rⁿ are each halogen.

In some embodiments, R^{m} and Rⁿ are each fluorine.

In some embodiments, one of R^{m} and Rⁿ is hydrogen and the other is halogen. In some embodiments of such compounds, the halogen and the phenyl ring are in a trans configuration with respect to one another on the cyclobutyl ring. In some embodiments of such compounds, the halogen and the phenyl ring are in a cis configuration with respect to one another on the cyclobutyl ring.

In some embodiments, one of R^{m} and Rⁿ is hydrogen and the other is fluorine. In some embodiments of such compounds, the fluorine and the phenyl ring are in a trans configuration with respect to one another on the cyclobutyl ring. In some embodiments of such compounds, the fluorine and the phenyl ring are in a cis configuration with respect to one another on the cyclobutyl ring.

In some embodiments, R⁷ is selected from phenyl, 2-fluorophenyl, 3-fluorophenyl, 2,4-difluorophenyl, 3,4-difluorophenyl, 3,5-difluorophenyl, 4-fluorophenyl, 2-chlorophenyl, 3-chlorophenyl, 4-chlorophenyl, 2, 4-dichlorophenyl, 3,4-dichlorophenyl, 3,5-dichlorophenyl, 2-methylphenyl, 3-methylphenyl, 2, 4-dimethylphenyl, 3,4-dimethylphenyl, 3,5-dimethylphenyl, 2-(hydroxymethyl)phenyl, 3-(hydroxymethyl)phenyl, 4-(hydroxymethyl)phenyl, 2-(aminomethyl)phenyl, 3-(aminomethyl)phenyl, 4-(aminomethyl)phenyl, 2-phenol, 3-phenol, 4-phenol, 2-methoxyphenyl, 3-methoxyphenyl, 4-methoxyphenyl, 2-difluoromethoxyphenyl, 3-difluoromethoxyphenyl, 4-difluoromethoxyphenyl, 2-trifluoromethoxyphenyl, 3-trifluoromethoxyphenyl, 4-trifluoromethoxyphenyl, 2-cyanophenyl, 3-cyanophenyl, 4-cyanophenyl, 2-benzamine, 3-benzamide, 4-benzamide, N-methyl-2-benzamine, N-methyl-3-benzamide, N-methyl-4-benzamide, N,N-dimethyl-2-benzamine, N,N-dimethyl-3-benzamide, and N, N-dimethyl-4-benzamide.

In some embodiments, R⁷ is 5-10 membered heteroaryl optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R_{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇11 aralkyl and 5-10 membered heteroaryl groups is optionally substituted with 1, 2, 3, 4 or 5 R^{f} substituents.

In some embodiments, R⁷ is pyridyl optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents.

In some embodiments, R⁷ is selected from 2-pyridyl, 3-pyridyl and 4-pyridyl, each optionally substituted with 1, 2, 3, 4 or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₆ cycloalkyl, C₃₋₆ cycloalkenyl, 3-6 membered heterocycloalkyl, 3-6 membered heterocycloalkenyl, phenyl, naphthyl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl and 5-10 membered heteroaryl groups is optionally substituted with 1, 2, 3, 4 or 5 R^{f} substituents.

In some embodiments, the compounds are of Formula IX, or a pharmaceutically acceptable salt thereof:

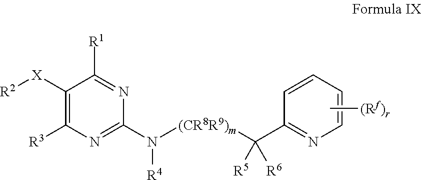

wherein r is 0, 1, 2, 3 or 4, and R¹, R², R³, R⁴, R⁵, R⁶, R⁸, R⁹, R^{f}, X and m are as defined herein.

In some embodiments, the compounds are of Formula X(a) or X(b), or a pharmaceutically acceptable salt thereof:

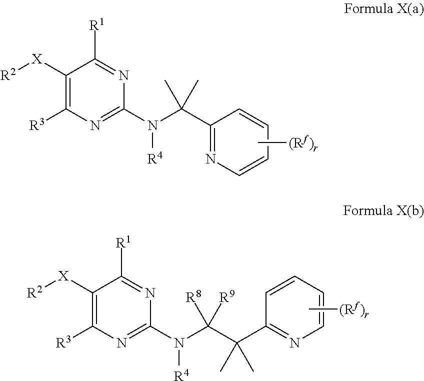

wherein r is 0, 1, 2, 3 or 4, and R¹, R², R³, R⁴, R⁸, R⁹, R^{f} and X are as defined herein.

In some embodiments, the compounds are of Formula XI(a) or XI(b), or a pharmaceutically acceptable salt thereof:

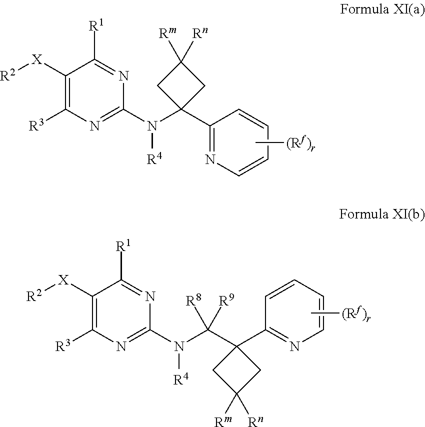

wherein R^{m} and Rⁿ are each independently selected from hydrogen, halogen and C₁₋₆ alkyl; r is 0, 1, 2, 3 or 4; and R¹, R², R³, R⁴, R⁸, R⁹, R^{f} and X are as defined herein.

In some embodiments, R^{m} and Rⁿ are each hydrogen.

In some embodiments, R^{m} and Rⁿ are each halogen.

In some embodiments, R^{m} and Rⁿ are each fluorine.

In some embodiments, one of R^{m} and Rⁿ is hydrogen and the other is halogen. In some embodiments of such compounds, the halogen and the pyridyl ring are in a trans configuration with respect to one another on the cyclobutyl ring. In some embodiments of such compounds, the halogen and the pyridyl ring are in a cis configuration with respect to one another on the cyclobutyl ring.

In some embodiments, one of R^{m} and Rⁿ is hydrogen and the other is fluorine. In some embodiments of such compounds, the fluorine and the pyridyl ring are in a trans configuration with respect to one another on the cyclobutyl ring. In some embodiments of such compounds, the fluorine and the pyridyl ring are in a cis configuration with respect to one another on the cyclobutyl ring.

In some embodiments, R⁷ is selected from pyrid-2-yl, 3-fluoro-pyrid-2-yl, 4-fluoro-pyrid-2-yl, 5-fluoro-pyrid-2-yl, 6-fluoro-pyrid-2-yl, 3-chloro-pyrid-2-yl, 4-chloro-pyrid-2-yl, 5-chloro-pyrid-2-yl, 6-chloro-pyrid-2-yl, 3-cyano-pyrid-2-yl, 4-cyano-pyrid-2-yl, 5-cyano-pyrid-2-yl, 6-cyano-pyrid-2-yl, 3-methyl-pyrid-2-yl, 4-methyl-pyrid-2-yl, 5-methyl-pyrid-2-yl, 6-methyl-pyrid-2-yl, 3-difluoromethyl-pyrid-2-yl, 4-difluoromethyl-pyrid-2-yl, 5-difluoromethyl-pyrid-2-yl, 6-difluoromethyl-pyrid-2-yl, 3-trifluoromethyl-pyrid-2-yl, 4-trifluoromethyl-pyrid-2-yl, 5-trifluoromethyl-pyrid-2-yl, 6-trifluoromethyl-pyrid-2-yl, 3-hydroxymethyl-pyrid-2-yl, 4-hydroxymethyl-pyrid-2-yl, 5-hydroxymethyl-pyrid-2-yl, 6-hydroxymethyl-pyrid-2-yl, 3-aminomethyl-pyrid-2-yl, 4-aminomethyl-pyrid-2-yl, 5-aminomethyl-pyrid-2-yl, 6-aminomethyl-pyrid-2-yl, 3-hydroxy-pyrid-2-yl, 4-hydroxy-pyrid-2-yl, 5-hydroxy-pyrid-2-yl, 6-hydroxy-pyrid-2-yl, 3-methoxy-pyrid-2-yl, 4-methoxy-pyrid-2-yl, 5-methoxy-pyrid-2-yl, 6-methoxy-pyrid-2-yl, 3-difluoromethoxy-pyrid-2-yl, 4-difluoromethoxy-pyrid-2-yl, 5-difluoromethoxy-pyrid-2-yl, 6-difluoromethoxy-pyrid-2-yl, 3-trifluoromethoxy-pyrid-2-yl, 4-trifluoromethoxy-pyrid-2-yl, 5-trifluoromethoxy-pyrid-2-yl, 6-trifluoromethoxy-pyrid-2-yl, 3-methylthio-pyrid-2-yl, 4-methylthio-pyrid-2-yl, 5-methylthio-pyrid-2-yl, 6-m ethylthio-pyrid-2-yl, 3-carboxamide-pyrid-2-yl, 4-carboxamide-pyrid-2-yl, 5-carboxamide-pyrid-2-yl, 6-carboxamide-pyrid-2-yl and 3-fluoro-6-methyl-pyrid-2-yl.

In some embodiments, R⁷ is selected from pyrid-3-yl, 2-fluoro-pyrid-3-yl, 4-fluoro-pyrid-3-yl, 5-fluoro-pyrid-3-yl, 6-fluoro-pyrid-3-yl, 2-chloro-pyrid-3-yl, 4-chloro-pyrid-3-yl, 5-chloro-pyrid-3-yl, 6-chloro-pyrid-3-yl, 2-cyano-pyrid-3-yl, 4-cyano-pyrid-3-yl, 5-cyano-pyrid-3-yl, 6-cyano-pyrid-3-yl, 2-methyl-pyrid-3-yl, 4-methyl-pyrid-3-yl, 5-methyl-pyrid-3-yl, 6-methyl-pyrid-3-yl, 2-difluoromethyl-pyrid-3-yl, 4-difluoromethyl-pyrid-3-yl, 5-difluoromethyl-pyrid-3-yl, 6-difluoromethyl-pyrid-3-yl, 2-trifluoromethyl-pyrid-3-yl, 4-trifluoromethyl-pyrid-3-yl, 5-trifluoromethyl-pyrid-3-yl, 6-trifluoromethyl-pyrid-3-yl, 2-hydroxymethyl-pyrid-3-yl, 4-hydroxymethyl-pyrid-3-yl, 5-hydroxymethyl-pyrid-3-yl, 6-hydroxymethyl-pyrid-3-yl, 2-aminomethyl-pyrid-3-yl, 4-aminomethyl-pyrid-3-yl, 5-aminomethyl-pyrid-3-yl, 6-aminomethyl-pyrid-3-yl, 2-hydroxy-pyrid-3-yl, 4-hydroxy-pyrid-3-yl, 5-hydroxy-pyrid-3-yl, 6-hydroxy-pyrid-3-yl, 2-methoxy-pyrid-3-yl, 4-methoxy-pyrid-3-yl, 5-methoxy-pyrid-3-yl, 6-methoxy-pyrid-3-yl, 2-difluoromethoxy-pyrid-3-yl, 4-difluoromethoxy-pyrid-3-yl, 5-difluoromethoxy-pyrid-3-yl, 6-difluoromethoxy-pyrid-3-yl, 2-trifluoromethoxy-pyrid-3-yl, 4-trifluoromethoxy-pyrid-3-yl, 5-trifluoromethoxy-pyrid-3-yl, 6-trifluoromethoxy-pyrid-3-yl, 2-methylthio-pyrid-3-yl, 4-methylthio-pyrid-3-yl, 5-methylthio-pyrid-3-yl, 6-methylthio-pyrid-3-yl, 2-carboxamide-pyrid-3-yl, 4-carboxamide-pyrid-3-yl, 5-carboxamide-pyrid-3-yl and 6-carboxamide-pyrid-3-yl.

In some embodiments, VI, VII(a), VII(b), VIII(a), VIII(b), IX, X(a), X(b), XI(a) or XI(b), X is selected from a bond, —(CH₂)ₚ—, —(CH₂)ₚO(CH₂)_{q}—, —(CH₂)ₚC(O)(CH₂)_{q}—, —(CH₂)ₚS(CH₂)_{q}—, —(CH₂)ₚNR^{d}(CH₂)_{q}—, —(CH₂)ₚC(O)O(CH₂)_{q}—, —(CH₂)ₚOC(O)(CH₂)_{q}—, —(CH₂)ₚNR^{d}C(O)(CH₂)_{q}—, —(CH₂)ₚC(O)NR^{d}(CH₂)_{q}—, —(CH₂)ₚNR^{d}C(O)NR^{d}(CH₂)_{q}—, —(CH₂)ₚNR^{d}SO₂(CH₂)_{q}—, and —(CH₂)ₚSO₂NR^{d}(CH₂)_{q}—.

In some embodiments, X is a bond.

In some embodiments, the compound is of Formula XII(a), XII(b), XII(c), XII(d), XII(e), XII(f), XII(g), XII(h), XII(i), XII(j), XII(k), XII(l), XII(m), XII(n) or XII(o), or a pharmaceutically acceptable salt thereof:

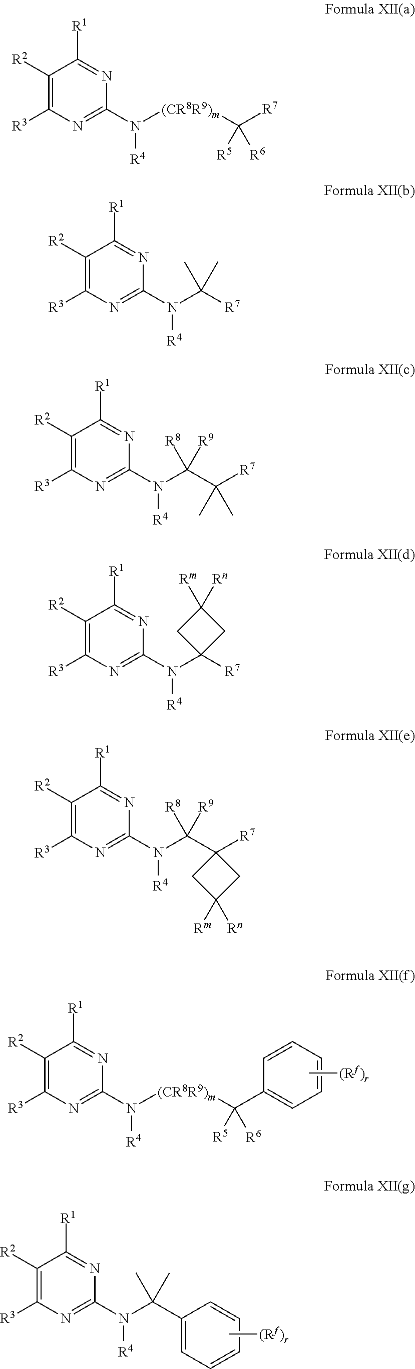

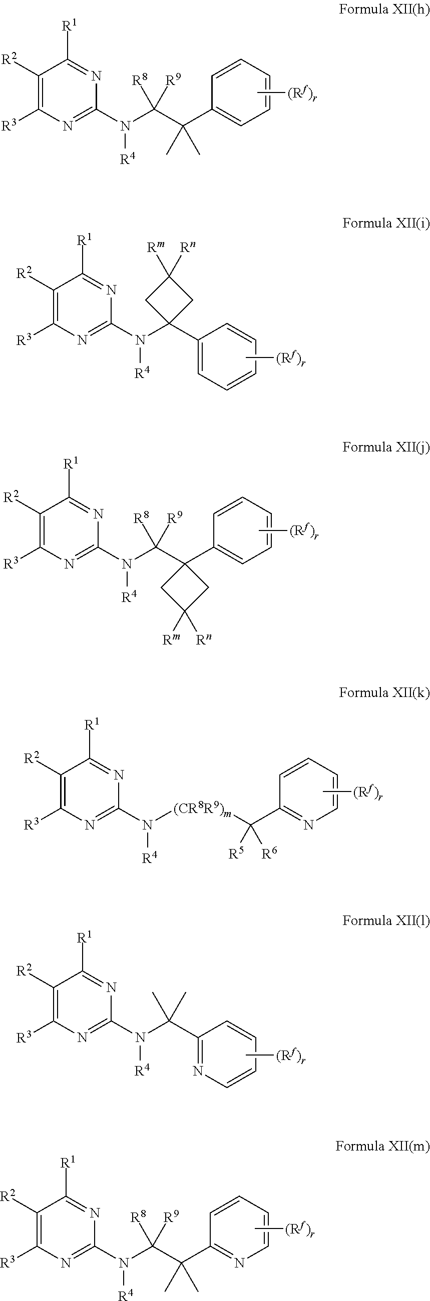

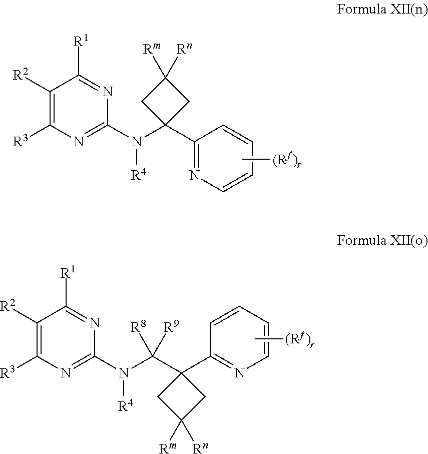

wherein R¹, R², R³, R⁴, R⁵, R⁶, R⁷, R⁸, R⁹, R^{f}, R^{m}, Rⁿ, m and r are as defined herein.

In some embodiments, X is —O—.

In some embodiments, X is selected from —CH₂O— and —OCH₂—.

In some embodiments, X is —NR^{d}—.

In some embodiments, X is selected from —CH₂NR^{d}— and —NR^{d}CH₂—.

In some embodiments, X is selected from —NR^{d}C(O)— and —C(O)NR^{d}—.

In some embodiments, X is selected from —CH₂NR^{d}C(O)— and —C(O)NR^{d}CH₂—.

In some embodiments, R² is selected from C₃₋₈ cycloalkyl, C₃₋₈ cycloalkenyl, 3-8 membered heterocycloalkyl, 3-8 membered heterocycloalkenyl, C₆₋₁₀ aryl and 5-10 membered heteroaryl, each optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is phenyl optionally substituted with 1, 2, 3, 4 or 5 substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is phenyl substituted with 1, 2, 3, 4 or 5 substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)NR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents; wherein at least one substitutent is bonded at the meta position.

In some, R² is phenyl substituted with a substituent selected from (CH₂)ₙC(O)OR^{a} and (CH₂)ₙC(O)NR^{b}R^{c}; and optionally substituted with 1, 2 or 3 additional substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is phenyl substituted with a substituent selected from C(O)OH, C(O)NH₂, C(O)OC₁₋₆ alkyl, C(O)NHC₁₋₆ alkyl and C(O)N(C₁₋₆ alkyl)₂; and optionally substituted with 1, 2 or 3 additional substituents selected from halogen, C₁₋₆ alkyl and C₁₋₆ haloalkyl.

In some embodiments, R² is phenyl substituted at the meta position with a substituent selected from (CH₂)ₙC(O)OR^{a} and (CH₂)ₙC(O)NR^{b}R^{c}; and optionally substituted with 1, 2 or 3 additional substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is phenyl substituted at the meta position with a substituent selected from (CH₂)ₙC(O)OR^{a} and (CH₂)ₙC(O)NR^{b}R^{c}, and optionally substituted with 1, 2 or 3 additional substituents selected from halogen, hydroxyl, C₁₋₆ alkoxy, CN, C₁₋₆ alkyl and C₁₋₆ haloalkyl.

In some embodiments, R² is phenyl substituted at the meta position with a substituent selected from C(O)OH, C(O)NH₂, C(O)OC₁₋₆ alkyl, C(O)NHC₁₋₆ alkyl and C(O)N(C₁₋₆ alkyl)₂; and optionally substituted with 1, 2 or 3 additional substituents selected from halogen, hydroxyl, C₁₋₆ alkoxy, CN, C₁₋₆ alkyl and C₁₋₆ haloalkyl.

In some embodiments, R² is phenyl substituted with (CH₂)ₙNR^{d}C(O)R^{a}, wherein R^{a} is C₁₋₆ alkyl or 3-8 membered heterocycloalkyl, each optionally substituted with 1, 2 or 3 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl; and optionally substituted with 1, 2 or 3 additional substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is phenyl substituted with (CH₂)ₙNR^{d}C(O)R^{a}, wherein R^{a} is selected from C₁₋₆ alkyl, C₁₋₆ alkyl-OH and C₁₋₆ alkyl-NH₂, each optionally substituted with 1, 2 or 3 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, and (CH₂)ₙ5-10 membered heteroaryl; and optionally substituted with 1, 2 or 3 additional substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)ₙNR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is 3-benzamide, N-methyl-3-benzamide, N,N-dimethyl-3-benzamide, 4-fluoro-3-benzamide, N-methyl-4-fluoro-3-benzamide, N,N-dimethyl-4-fluoro-3-benzamide, 3-benzoic acid, methyl-3-benzoate, 4-fluoro-3-benzoic acid and methyl-4-fluoro-3-benzoate.

In some embodiments, R² is 5-10 membered heteroaryl optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is selected from pyridyl, pyrimidyl, pyrazyl, pyridazyl, triazyl, furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with 1, 2, 3 or 4 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is selected from pyridyl, pyrimidyl, pyrazyl, pyridazyl, triazyl, furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with a substituent selected from (CH₂)ₙC(O)OR^{a} and (CH₂)ₙC(O)NR^{b}R^{c}; and optionally substituted with 1, 2 or 3 additional substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is selected from pyridyl, pyrimidyl, pyrazyl, pyridazyl and triazyl, each optionally substituted with (CH₂)ₙC(O)NR^{b}R^{c}.

In some embodiments, R² is selected from furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with (CH₂)ₙC(O)NR^{b}R^{c}.

In some embodiments, R² is selected from pyridyl, pyrimidyl, pyrazyl, pyridazyl and triazyl, each optionally substituted with (CH₂)ₙC(O)NH₂.

In some embodiments, R² is selected from furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with (CH₂)ₙC(O)NH₂.

In some embodiments, R² is selected from pyridyl, pyrimidyl, pyrazyl, pyridazyl, triazyl, furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with (CH₂)ₙNR^{d}C(O)R^{a}, wherein R^{a} is C₁₋₆ alkyl or 3-8 membered heterocycloalkyl, each optionally substituted with 1, 2 or 3 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is selected from pyridyl, pyrimidyl, pyrazyl, pyridazyl and triazyl, each optionally substituted with (CH₂)ₙNR^{d}C(O)R^{a}, wherein R^{a} is selected from C₁₋₆ alkyl, C₁₋₆ alkyl-OH and C₁₋₆ alkyl-NH₂, each optionally substituted with 1, 2 or 3 substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl.

In some embodiments, R² is selected furanyl, pyrrolyl, thiophenyl, thiazolyl, isothiazolyl, thiadiazolyl, oxazolyl, isoxazolyl, oxadiazolyl, imidazolyl, triazolyl and tetrazolyl, each optionally substituted with (CH₂)ₙNR^{d}C(O)R^{a}, wherein R^{a} is selected from C₁₋₆ alkyl, C₁₋₆ alkyl-OH and C₁₋₆ alkyl-NH₂, each optionally substituted with 1, 2 or 3 substituents selected from halogen, CN, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl.

In some embodiments, R² is selected from indolyl, indazolyl, benzimidazolyl, benzoxazolyl and benzoisoxazolyl, each optionally substituted with 1, 2, 3 or 4 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is selected from 1H-indazol-6-yl, 1H-indazol-5-yl, 1H-indazol-4-yl, 3-amino(1H-indazol-5-yl), 3-amino(1H-indazol-6-yl), 3-amino(1H-indazol-7-yl), 1-methyl(1H-indazol-6-yl), 3-methyl(1H-indazol-6-yl), 3-amino-1-methyl(1H-indazol-5-yl), 3-cyano(1H-indazol-5-yl), 3-carboxamide(1H-indazol-5-yl), 3-carboxamidine(1H-indazol-5-yl), 3-vinyl(1H-indazol-5-yl), 3-ethyl(1H-indazol-5-yl), 3-acetamide(1H-indazol-5-yl), 3-methylsulfonylamine(1H-indazol-5-yl), 3-methoxycarboxamide(1H-indazol-5-yl), 3-methylamino(1H-indazol-5-yl), 3-dimethylamino(1H-indazol-5-yl), 3-ethylamino(1H-indazol-5-yl), 3-(2-aminoethyl)amino(1H-indazol-5-yl), 3-(2-hydroxyethyl)amino(1H-indazol-5-yl), 3-[(methylethyl)amino](1H-indazol-5-yl), 6-benzimidazol-5-yl, 6-(2-methylbenzimidazol-5-yl), 2-aminobenzimidazol-5-yl, 2-hydroxybenzimidazol-5-yl, 2-acetamidebenzimidazol-5-yl, 3-aminobenzo[3,4-d]isoxazol-5-yl, 3-aminobenzo[d]isoxazol-6-yl, 3-aminobenzo[d]isoxazol-7-yl, 2-methylbenzoxazol-5-yl and 2-methylbenzoxazol-6-yl.

In some embodiments, R² is selected from 3-6 membered heterocycloalkyl and 3-6 membered heterocycloalkenyl, each optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is selected from aziridinyl, azetidinyl, pyrrolidinyl, piperidinyl, piperazinyl and morpholinyl, each optionally substituted with 1, 2, 3, 4 or 5 substituents selected from halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)ₙNR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)ₙC(S)R^{a}, (CH₂)ₙC(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)ₙSO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙphenyl, (CH₂)ₙnaphthyl and (CH₂)ₙ5-10 membered heteroaryl groups is optionally substituted with 1, 2, 3, 4 or 5 R^{f} substituents.

In some embodiments, R² is NR^{b}R^{c}, wherein R^{b} and R^{c} are as defined herein.

In some embodiments, R² is NR^{b}R^{c}, wherein one of R^{b} and R^{c} is hydrogen and the other is C₁₋₆ alkyl optionally substituted with 1, 2, 3, 4 or 5 R^{f} substituents.

In some embodiments, X is —C(O)— and R² is NR^{b}R^{c}, wherein R^{b} and R^{c} are as defined herein.

In some embodiments, X is —C(O)— and R² is NR^{b}R^{c}, wherein one of R^{b} and R^{c} is hydrogen and the other is C₁₋₆ alkyl optionally substituted with 1, 2, 3, 4 or 5 R^{f} substituents.

In some embodiments, X is —(CH₂)ₚ— and R² is NR^{b}R^{c}, wherein R^{b} and R^{c} are as defined herein.

In some embodiments, X is —(CH₂)ₚ— and R² is NR^{b}R^{c}, wherein one of R^{b} and R^{c} is hydrogen and the other is C₁₋₆ alkyl optionally substituted with 1, 2, 3, 4 or 5 R^{f} substituents.

In some embodiments, X, R² and R³, together with the carbon atoms to which they are bound, form a 5-6 membered ring optionally containing one or more heteroatoms selected from oxygen nitrogen and sulfur, and optionally containing one or more double bonds, and optionally substituted with 1, 2, 3, 4 or 5 R^{f} substituents.

In some embodiments, the compound is of Formula XIII, or a pharmaceutically acceptable salt thereof:

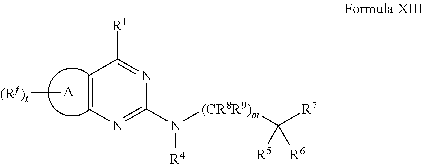

wherein A is a 5 or 6 membered ring optionally containing one or more heteroatoms selected from oxygen nitrogen and sulfur, and optionally containing one or more double bonds; t is 0, 1, 2, 3 or 4; and R¹, R⁴, R⁵, R⁶, R⁷, R⁸, R⁹, R^{f} and m are as defined herein.

In some embodiments, ring A together with the pyrimidine ring to which it is bound form a group selected from quinazoline, pyrido[2,3-d]pyrimidine, pyrido[3,4-d]pyrimidine, pyrido[4,3-d]pyrimidine, pyrido[3,2-d]pyrimidine, 5,6,7,8-tetrahydroquinazoline, 5,6,7,8-tetrahydropyrido[2,3-d]pyrimidine, 5,6,7,8-tetrahydropyrido[3,4-d]pyrimidine, 5,6,7,8-tetrahydropyrido[4,3-d]pyrimidine, 5,6,7,8-tetrahydropyrido[3,2-d]pyrimidine, thieno[3,2-d]pyrimidine, thiazolo[4,5-d]pyrimidine, 5H-pyrrolo[3,2-d]pyrimidine, 7H-purine, thieno[2,3-d]pyrimidine, thiazolo[5,4-d]pyrimidine, 7H-pyrrolo[2,3-d]pyrimidine, 9H-purine, 1H-pyrazolo[4,3-d]pyrimidine, 1H-pyrazolo[3,4-d]pyrimidine, 1H-[1,2,3]triazolo[4,5-d]pyrimidine, 3H-[1,2,3]triazolo[4,5-d]pyrimidine, 6,7-dihydro-5H-pyrrolo[2,3-d]pyrimidine, 6,7-dihydro-5H-pyrrolo[3,4-d]pyrimidine, 6,7-dihydro-5H-pyrrolo[3,2-d]pyrimidine and 6,7-dihydro-5H-cyclopenta[d]pyrimidine, each optionally substituted with 1, 2, 3, 4 or 5 R^{f} substituents.

In some embodiments, Ring A together with the pyrimidine ring to which it is bound form a group selected from quinazoline, 5,6,7,8-tetrahydropyrido[4,3-d]pyrimidine, 5,6,7,8-tetrahydropyrido[3,4-d]pyrimidine, 1H-pyrazolo[3,4-d]pyrimidine, thieno[2,3-d]pyrimidine and thiazolo[5,4-d]pyrimidine, each optionally substituted with 1, 2, 3, 4 or 5 R^{r} substituents.

In some embodiments, R¹ is selected from hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl and 5-10 membered heteroaryl.

In some embodiments, R¹ is selected from hydrogen, halogen, CN, C₁₋₆ alkyl, C₁₋₆ haloalkyl, hydroxyl, C₁₋₆ alkoxy, NH₂, NHC₁₋₆ alkyl, and N(C₁₋₆ alkyl)₂.

In some embodiments, R¹ is selected from hydrogen, halogen, CN, CF₃ and methyl.

In some embodiments, R¹ is hydrogen.

In some embodiments, R³ is selected from hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl and 5-10 membered heteroaryl.

In some embodiments, R³ is selected from hydrogen, halogen, CN, C₁₋₆ alkyl, C₁₋₆ haloalkyl, hydroxyl, C₁₋₆ alkoxy, NH₂, NHC₁₋₆ alkyl, and N(C₁₋₆ alkyl)₂.

In some embodiments, R³ is selected from hydrogen, halogen, CN, CF₃ and methyl.

In some embodiments, R³ is hydrogen.

In some embodiments, R¹ and R³ are each hydrogen.

In some embodiments, R⁴ is selected from hydrogen, C₁₋₆ alkyl, C₁₋₆ haloalkyl, C(O)R^{a}, C(O)OR^{a}, C(O)NR^{b}R^{c} and SO₂R^{a}.

In some embodiments, R⁴ is hydrogen.

In some embodiments, R¹, R³ and R⁴ are each hydrogen.

In some embodiments, R⁸ and R⁹, at each occurrence, are each independently selected from hydrogen, halogen and C₁₋₆ alkyl.

In some embodiments, R⁸ and R⁹, at each occurrence, are each hydrogen.

In some embodiments, a compound of Formula I is 1-(2-((3-fluoro-1-(3-fluoropyridin-2-yl)cyclobutyl)methylamino)pyrimidin-5-yl)-1H-pyrrole-3-carboxamide or a pharmaceutically acceptable salt thereof. In some embodiments, a compound of Formula I is 1-(2-(((trans)-3-fluoro-1-(3-fluoropyridin-2-yl)cyclobutyl)methylamino)pyrimidin-5-yl)-1H-pyrrole-3-carboxamide (Compound D) or a pharmaceutically acceptable salt thereof. In some embodiments, a compound of Formula I is 3-(2-((-3-fluoro-1-(3-fluoropyridin-2-yl)cyclobutyl)methylamino)pyrimidin-5-yl)benzamide or a pharmaceutically acceptable salt thereof. In some embodiments, a compound of Formula I is 3-(2-(((trans)-3-fluoro-1-(3-fluoropyridin-2-yl)cyclobutyl)methylamino)-pyrimidin-5-yl)benzamide (Compound B) or a pharmaceutically acceptable salt thereof.

Methods of preparing compounds described herein are readily available in the art. U.S. Pat. No. 7,956,056, for instance, discloses methods of preparing compounds of Formula A and Formula B. WO 2011/133888, furthermore, provides synthesis methods for Formulas I-XIII. The contents of these patents and patent applications are incorporated into the present disclosure by reference in their entirety.

It is also contemplated that skeletal muscle troponin activators suitable for methods described herein can be compounds disclosed in U.S. Pat. Nos. 8,227,603, 8,063,082, 7,956,056, 7,851,484, 7,598,248 and 7,989,469, and PCT Publication Nos. WO/2013/010015, WO/2008/016648, WO/2009/099594, WO/2011/0133920, WO/2011/133888, WO/2011/133882, and WO/2011/13392. The contents of these patents and patent applications are incorporated into the present disclosure by reference in their entirety. In some embodiments, the skeletal muscle troponin activator is 1-((1R)-1-methylpropyl)-6-chloro-7-pyrazolylimidazo[4,5-b]pyridin-2-ol or a pharmaceutically acceptable salt thereof.

The chemical entities described herein are administered at a therapeutically effective dosage, e.g., a dosage sufficient to provide treatment for the disease states previously described. While human dosage levels have yet to be optimized for the chemical entities described herein, generally, a daily dose ranges from about 0.05 to 100 mg/kg of body weight; in certain embodiments, from about 0.10 to 10.0 mg/kg of body weight, and in certain embodiments, from about 0.15 to 1.0 mg/kg of body weight. Thus, for administration to a 70 kg person, in certain embodiments, the dosage range would be about from 3.5 to 7000 mg per day; in certain embodiments, about from 7.0 to 700.0 mg per day, and in certain embodiments, about from 10.0 to 100.0 mg per day. The amount of the chemical entity administered will, of course, be dependent on the subject and disease state being treated, the severity of the affliction, the manner and schedule of administration and the judgment of the prescribing physician; for example, a likely dose range for oral administration would be from about 70 to 700 mg per day, whereas for intravenous administration a likely dose range would be from about 70 to 700 mg per day depending on compound pharmacokinetics.

Administration of the chemical entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used.

Pharmaceutically acceptable compositions include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

The following examples serve to more fully describe the disclosed compounds the methods. It is understood that these examples in no way serve to limit the true scope of this invention, but rather are presented for illustrative purposes.

### EXAMPLE 1: GENERAL METHOD FOR FORCE-PCA SKINNED MUSCLE FIBER ANALYSIS

This example demonstrates the preparation of skinned muscle fibers and the use of these fibers to study the function of fast skeletal muscle troponin activators on muscle (e.g., diaphragm muscle) fibers.

Muscle tissue for in vitro skinned fiber studies were prepared using a protocol based on Lynch and Faulkner (Am J Physiol 275:C1548-54 (1998)). Briefly, rat diaphragm or rabbit psoas muscles were rapidly dissected and rinsed with physiological saline. Muscles were then incubated in skinning solution (125 mM K-propionate, 20 mM imidazole, 5 mM EGTA, 2 mM MgCl₂, 2 mM ATP, pH 7.0) supplemented with 0.5% Brij 58 (Sigma Chemicals, St. Louis, Mo.) or 0.5% Triton X-100 (Sigma Chemicals, St. Louis, Mo.) for 30 minutes at 4° C. Muscles were then placed in storage solution (125 mM K-propionate, 20 mM imidazole, 5 mM EGTA, 2 mM MgCl₂, 2 mM ATP, glycerol 50%, pH 7.0) at −20° C. Muscles were incubated in storage solution at −20° C. for later use.

For skinned fiber analysis, single muscle fibers were dissected from larger segments of tissue in rigor buffer at 4° C. (20 μM MOPS, 5 μM MgCl2, 120 μM potassium acetate, 1 μM EGTA, pH 7.0). They were then suspended between a 400 A force transducer (Aurora Scientific, Ontario, Canada) and a fixed post and secured with 2-4 μl of a 5% solution of methylcellulose in acetone. Fibers were then incubated at 10° C. in a relaxing buffer (20 μM MOPS, 5.5 μM MgCl₂, 132 μM potassium acetate, 4.4 μM ATP, 22 μM creatine phosphate, 1 mg/ml creatine kinase, 1 mM DTT, 44 ppm antifoam, pH 7.0) and baseline tension adjusted. Tension was generated in each fiber by changing fiber buffer over to relax buffer supplemented with 1 mM EGTA and one or more concentrations of aqueous calcium chloride. Test articles were added to these buffers in a 1% DMSO solution.

### EXAMPLE 2: FORCE-PCA SKINNED MUSCLE FIBER ANALYSIS OF COMPOUND A

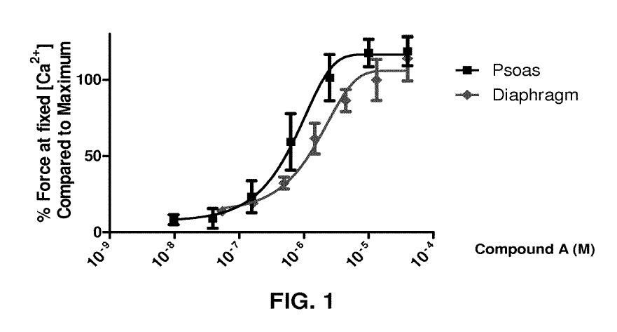

The functional effects of the fast skeletal muscle troponin activator, Compound A (6-bromo-1-(ethylpropyl)imidazo[4,5-b]pyrazin-2-ol), on skeletal muscle force were assessed under isometric conditions in permeabilized single fibers from rabbit psoas muscle and rat diaphragm muscle according to the procedure of Example 1. Using a 15 μM calcium chloride solution, a final concentration of 3.16 μM free calcium ions (pCa=5.5) was achieved with the diaphragm muscle, while the free calcium concentration was 1.78 μM (pCa=5.75) with psoas muscle (calcium concentrations calculated using the web resource (www.stanford.edu/˜cpatton/webmaxc/webmaxcS.htm). Psoas muscle consists almost entirely of fast fibers. Because the muscle membranes are removed in the preparation of the tissues, contractile force can be measured after direct application of calcium. As shown in FIG. 1, Treatment of skinned psoas or diaphragm fibers with Compound A (10 nM-40 μM) revealed dose-dependent increases in fiber sensitivity at a constant concentration of calcium. For psoas muscle, the EC₅₀ was found to be 0.59 μM (n=3) and the EC₅₀ for rat diaphragm was found to be 1.2 μM (n=4).

As shown in FIG. 1, Compound A increased tension in rat diaphragm muscle and rabbit psoas muscle.

### EXAMPLE 3: FORCE-PCA SKINNED MUSCLE FIBER ANALYSIS OF COMPOUND B

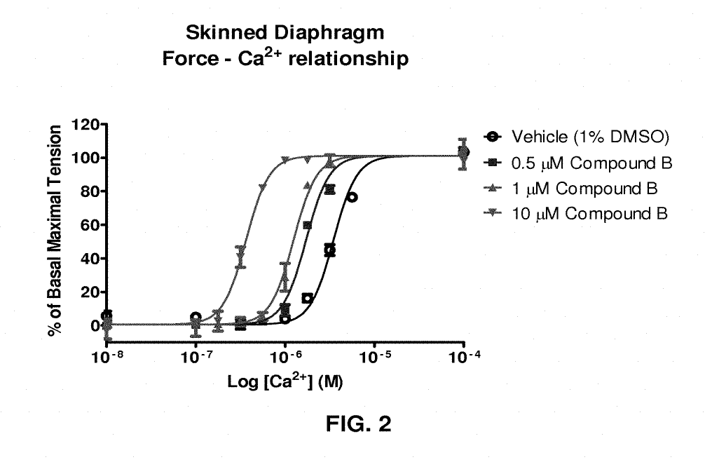

Force production was measured in rat skinned diaphragm fibers exposed to increasing concentrations of calcium in the presence and absence of the fast skeletal muscle troponin activator Compound B, (3-(2-(((trans)-3-fluoro-1-(3-fluoropyridin-2-yl)cyclobutyl)methylamino)pyrimidin-5-yl)benzamide), according to the procedure of Example 1. The results of this experiment are presented in FIG. 2 and Table 1, below.

TABLE 1
pCa at 50% of Maximum Tension

| | Log [Ca²⁺] (M) |
|---|---|
| Vehicle | −5.43 ± 0.03 |
| 0.5 uM | −5.78 ± 0.008 |
| 1 uM | −5.90 ± 0.03 |
| 10 uM | −6.46 ± 0.03 |

As shown in Table 1 and FIG. 2, Compound B dose-dependently increased the calcium sensitivity of rat skinned diaphragm fibers. Muscle fibers treated with 10 μM Compound B exhibited a 10-fold increase in calcium sensitivity compared to vehicle-only muscle fibers.

### EXAMPLE 4: FORCE-PCA SKINNED MUSCLE FIBER ANALYSIS OF COMPOUND C

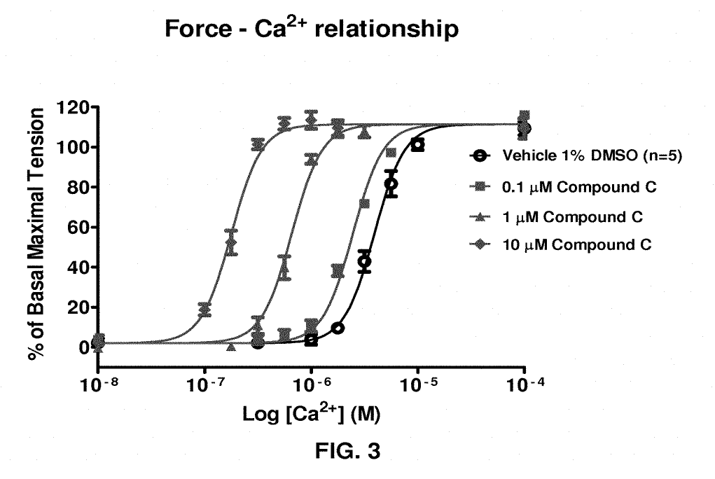

Force production was measured in rat skinned diaphragm fibers exposed to increasing concentrations of calcium in the presence and absence of the fast skeletal muscle troponin activator Compound C, 1-(ethylpropyl)-6-ethynylimidazo[4,5-b]pyrazin-2-ol, according to the procedure in Example 1. The results of this experiment are presented in FIG. 3 and Table 2, below.

TABLE 2
pCa at 50% of Maximum Tension

| | Log [Ca²⁺] (M) |
|---|---|
| Vehicle | −5.43 ± 0.054 |
| 0.1 uM | −5.59 ± 0.007 |
| 1 uM | −6.19 ± 0.018 |
| 10 uM | −6.74 ± 0.28 |

As shown in Table 2 and FIG. 3, Compound C dose-dependently increased the calcium sensitivity of rat skinned diaphragm fibers. Muscle fibers treated with 10 μM Compound C exhibited a 10-fold increase in calcium sensitivity compared to vehicle-only muscle fibers.

### EXAMPLE 5: DIAPHRAGM CHARACTERISTICS IN A RAT MODEL OF HEART FAILURE (HF)

Heart failure has a deleterious effect on respiratory function. It was hypothesized that the diaphragm, as a primary muscle involved in respiration, would be affected by heart failure and that a fast skeletal muscle troponin activator could improve its function.

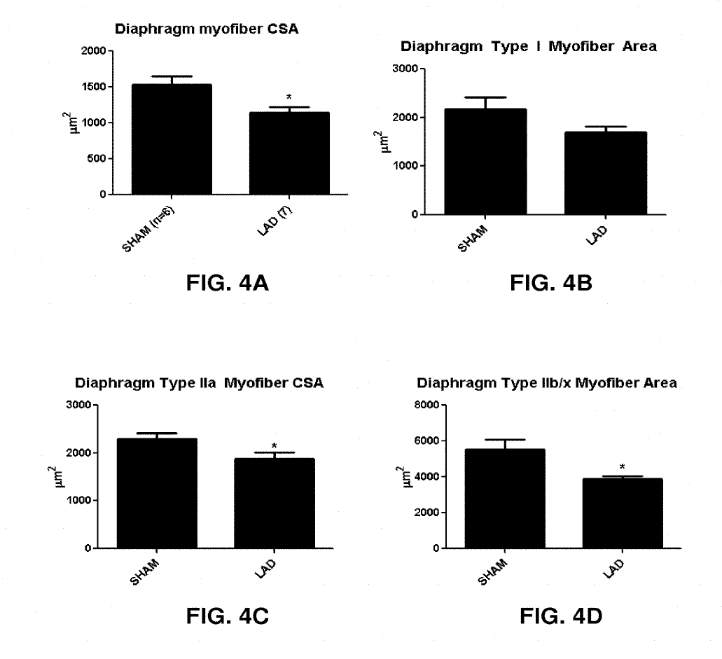

In this experiment, the effects of a HF on the diaphragm were studies using rats a rat model where the left anterior descending (LAD) coronary artery was ligated. The diaphragms from SHAM and LAD rats were excised, cleaned, pinned to corkboard, and frozen in melting isopentane. Serial frozen cross sections were cut at 10 μm and stained for myosin ATPase after preincubation at pH 4.35. Digital images were obtained under 200× total magnification (Olympus BX41) and analyzed by Axiovision software (Zeiss). Stained fibers were classified Type I, Type IIa, or Type II b/x and measured for individual myofiber cross-sectional area (μm²). The fiber type distribution of the SHAM and LAD rats is summarized in Table 3 and FIGS. 4A-4D (note: in the graphs, * indicates p<0.05).

TABLE 3

| | SHAM | LAD |
|---|---|---|
| % Type I | 35.3 ± 2.5 | 40 ± 2.5 |
| % Type IIa | 34.1 ± 3.5 | 30.8 ± 1.9 |
| % Type II b/x | 30.4 ± 2.6 | 29.1 ± 2.1 |

The experiment showed that mean diaphragm cross sectional area was significantly lower in HF diaphragm muscle. Within individual fiber types, significant atrophy in type IIa and type IIb/x fibers was observed in HF diaphragms. Fiber type distribution characterized by myosin ATPase activity was not significantly different between SHAM and HF animals.

### EXAMPLE 6: FORCE-FREQUENCY RELATIONSHIP ANALYSIS IN A RAT HF MODEL

Diaphragm contractile force was measured by electrical field stimulation in an organ bath system (Radnoti) based on a standard operating protocol adapted from the Treat NMD website (http://www.treat-nmd.eu/downloads/file/sops/dmd/MDX/DMD M.1.2.002.pdf). The diaphragm and the last floating rib from SHAM and LAD rats were excised, rinsed in physiological saline, and placed in a temperature controlled water-jacketed chamber (26-27° C.) containing Krebs-Henseleit Buffer (118 mM NaCl, 10 mM glucose, 4.6 mM KCl, 1.2 mM KH₂PO₄, 1.2 mM MgSO₄*7H₂O, 24.8 mM NaHCO₃, 2.5 mM CaCl₂, 50 mg/L tubocurarine, 50 U/L insulin, pH:7.4) that was continuously aerated with 95% O₂/5% O₂. After 10 minutes of equilibration, vertical strips spanning the floating rib to the central tendon were cut from diaphragms. Braided silk sutures were tied at the central tendon and floating rib and attached to a force transducer between two platinum electrodes. Diaphragm strips were set to a length that produced maximum twitch tension (Lo). The force-frequency profile of the muscle was obtained by stimulating the muscle at frequencies between 10-150 Hz (Grass Stimulator, 800 ms train duration, 0.6 ms pulse width).

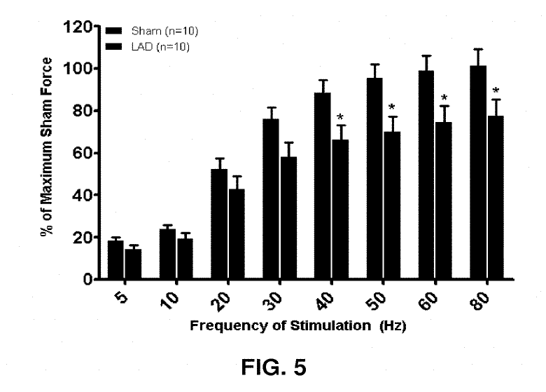

FIG. 5 shows that diaphragms from LAD animals exhibited less force output than those in from SHAM animals.

### EXAMPLE 7: FORCE-FREQUENCY RELATIONSHIP ANALYSIS OF COMPOUND B

Force production was measured in rat diaphragm muscle ex vivo by electrical field stimulation in the presence and absence of Compound B according to the procedure of Example 6. Compound B was suspended in DMSO and added directly to the bath.

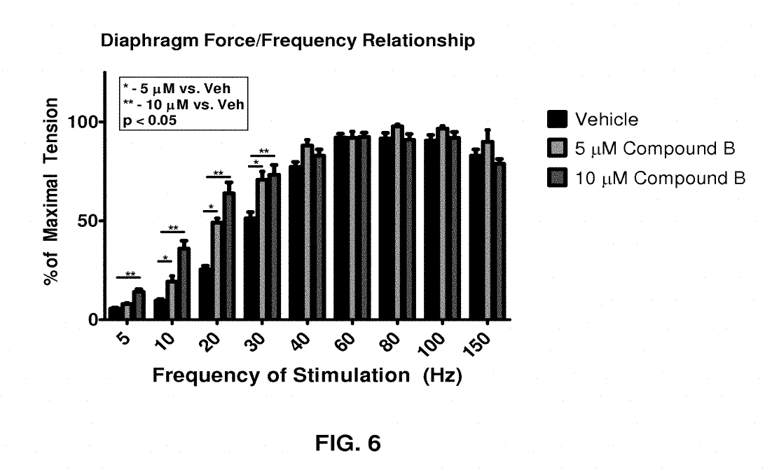

As shown in FIG. 6, diaphragm muscle treated with Compound B produced significantly more force compared to vehicle-only diaphragms at frequencies up to 30 Hz of electrical stimulation.

### EXAMPLE 8: DIAPHRAGM REPEATED CONTRACTION FATIGUE ANALYSIS

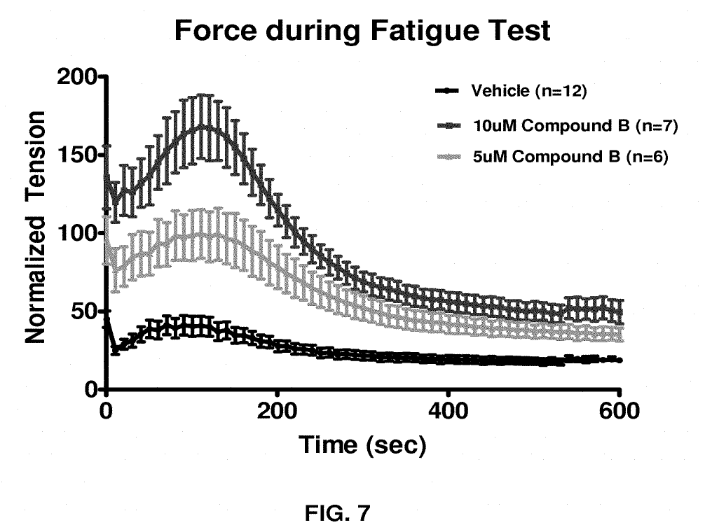

Following the procedure of Example 7, diaphragms were subjected to repeated electrical stimulations (20 Hz stimulation, 330 ms train duration, 1 train/sec) over a period of 10 minutes. Force production was measured over 600 contractions in rat diaphragm muscle ex vivo by field electrical stimulation in the presence and absence of Compound B (5 uM and 10 uM). As shown in FIG. 7, diaphragm muscle treated with Compound B produced significantly more force compared to vehicle-only diaphragms in a dose-dependent manner.

### EXAMPLE 9: FORCE-FREQUENCY RELATIONSHIP ANALYSIS OF COMPOUND D

Force production was measured in rat diaphragm muscle ex vivo by electrical field stimulation in the presence and absence of fast skeletal muscle troponin activator Compound D, 1-(2-(((trans)-3-fluoro-1-(3-fluoropyridin-2-yl)cyclobutyl)methylamino)pyrimidin-5-yl)-1H-pyrrole-3-carboxamide according to the procedure of Example 6. Compound D was suspended in DMSO and added directly to the bath.

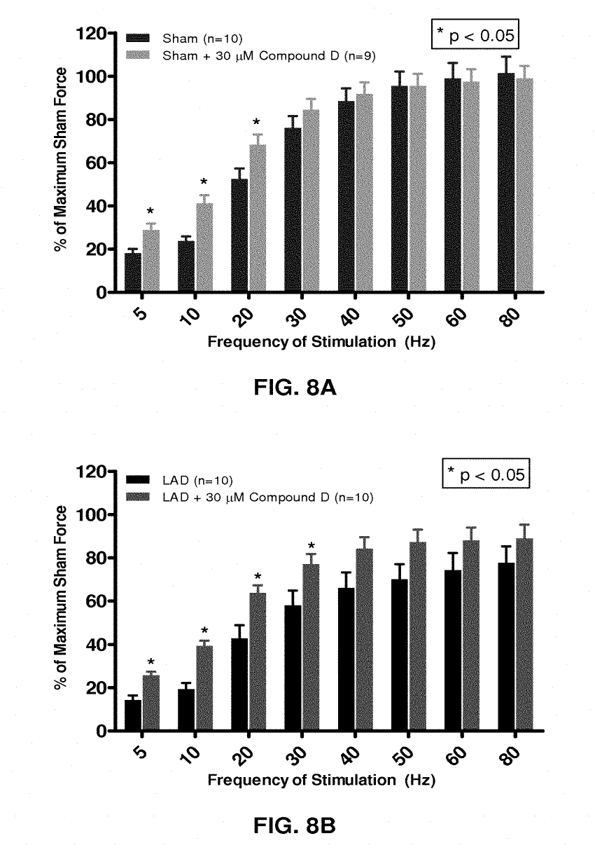

As shown in FIG. 8A and FIG. 8B, 30 μM Compound D significantly increased force in both SHAM and HF diaphragms at submaximal frequencies of electrical stimulation.

### EXAMPLE 10: FORCE-PCA SKINNED MUSCLE FIBER ANALYSIS OF COMPOUND D IN A RAT MODEL OF HF

Force production was measured in skinned diaphragm fibers from SHAM and LAD rats exposed to increasing concentrations of calcium in the presence and absence of Compound D according to the procedure of Example 1.

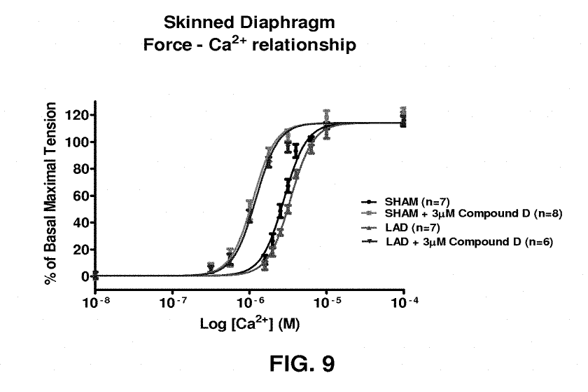

As shown in FIG. 9, HF diaphragm fibers have significantly lower Ca²⁺ sensitivity than SHAM fibers. Compound D (3 μM) significantly increased Ca²⁺ sensitivity in both SHAM and HF diaphragm fibers.

### EXAMPLE 11: DIAPHRAGM FORCE-FREQUENCY RELATIONSHIP ANALYSIS IN A MOUSE ALS Model

Respiratory weakness is a complication of ALS. It was hypothesized that a fast skeletal muscle troponin activator could increase the force output of the diaphragm of a subject suffering from ALS. To test this hypothesis, the SOD1 transgenic mouse, a rodent model of ALS, was used in this experiment.

Diaphragm contractile force was measured by electrical field stimulation in an organ bath system (Radnoti) based on a standard operating protocol adapted from the Treat NMD website (http://www.treat-nmd.eu/downloads/file/sops/dmd/MDX/DMD M.1.2.002.pdf). The diaphragm and the last floating rib from wild type (WT) and SOD1 mice were excised, rinsed in physiological saline, and placed in a temperature controlled water-jacketed chamber (26-27° C.) containing Krebs-Henseleit Buffer (118 mM NaCl, 10 mM glucose, 4.6 mM KCl, 1.2 mM KH₂PO₄, 1.2 mM MgSO₄*7H₂O, 24.8 mM NaHCO₃, 2.5 mM CaCl₂, 50 mg/L tubocurarine, 50 U/L insulin, pH:7.4) that was continuously aerated with 95% O₂/5% O₂. After 10 minutes of equilibration, vertical strips spanning the floating rib to the central tendon were cut from diaphragms. Braided silk sutures were tied at the central tendon and floating rib and attached to a force transducer between two platinum electrodes. Diaphragm strips were set to a length that produced maximum twitch tension (Lo). The force-frequency profile of the muscle was obtained by stimulating the muscle at frequencies between 10-150 Hz (Grass Stimulator, 800 ms train duration, 0.6 ms pulse width). Compound C was suspended in DMSO and directly added into the bath.

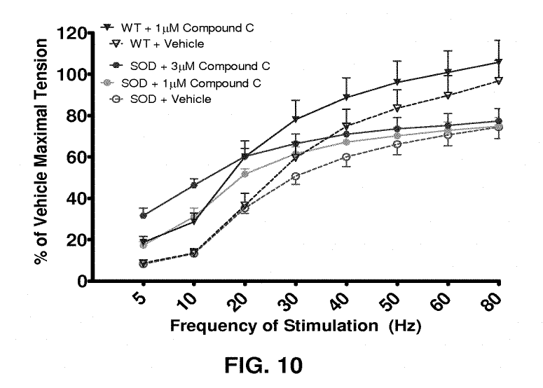

As shown in FIG. 10, Compound C increases submaximal force output in WT and SOD1 mouse diaphragm muscle in a dose-dependent manner. A trend for reduced force at higher frequencies of stimulation was observed in SOD1 diaphragm muscle. Both WT and SOD1 diaphragm muscle treated with Compound C produced significantly more force compared to vehicle-only diaphragms at frequencies up to 30 Hz of electrical stimulation.

### EXAMPLE 12: UNRESTRAINED WHOLE BODY PLETHYSMOGRAPHY (UWBP)

Wild type (WT) and SOD1 mice were dosed with vehicle or 10 mg/kg Compound C and placed in plethysmography chambers for 30 minutes of acclimatization. After acclimatization, respiratory parameters, including tidal volume, respiratory rate, and minute ventilation, were monitored for 10 minutes at room air. Upon completion of baseline room air measurements, animals were exposed to a 5% CO₂ gas mixture for 30 minutes. After the 5% CO₂ exposure, animals were re-exposed to room air and monitored.

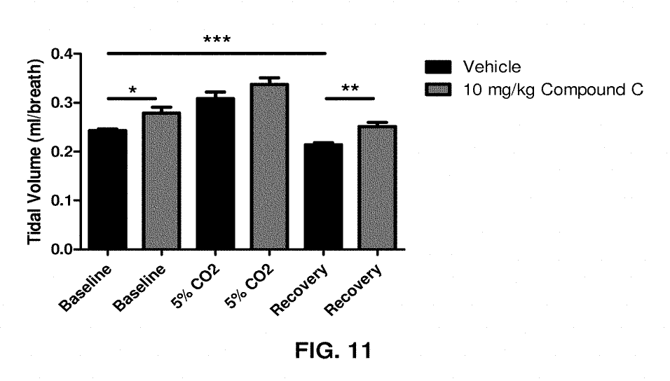

As shown in FIG. 11, compared to vehicle-treated animals, Compound C treated animals had significantly higher tidal volume at baseline and at recovery after a 30 minute exposure to a 5% CO₂ gas mixture.

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

1: A method for improving diaphragm function in a patient in need thereof, comprising administering to the patient an effective amount of a skeletal muscle troponin activator.

2: A method for increasing the function, activity, efficiency, sensitivity to calcium, or time to fatigue of skeletal muscle in the diaphragm of a patient in need thereof, comprising administering to the patient an effective amount of a skeletal muscle troponin activator.

3-4. (canceled)

5: The method of claim 1, wherein the patient is in use of mechanical ventilation.

6: The method of claim 1, wherein the patient undertakes an intense physical activity or is in an environment with a reduced partial pressure of oxygen in the air.

7: The method of claim 1, wherein the patient has a forced vital capacity (FVC) lower than about 75% of predicted of healthy individual in similar conditions, or the patient shows evidence of increased work of breathing indicative of reduced diaphragm function.

8: A method for increasing the function, activity, efficiency, force, sensitivity to calcium, or time to fatigue of a diaphragm skeletal muscle fiber, comprising contacting the fiber with an effective amount of a skeletal muscle troponin activator.

9: The method of claim 1, wherein the skeletal muscle is a fast skeletal muscle.

10: The method of claim 30, wherein the skeletal muscle troponin activator is a compound of Formula A or a compound of Formula B: 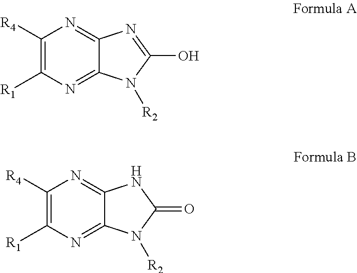
or a pharmaceutically acceptable salt thereof, wherein:
R₁ is alkenyl or alkynyl;
R₄ is hydrogen; and
R₂ is selected from the group consisting of 3-pentyl, 4-heptyl, 4-methyl-1-morpholinopentan-2-yl, isobutyl, cyclohexyl, cyclopropyl, sec-butyl, tert-butyl, isopropyl, 1-hydroxybutan-2yl, tetrahydro-2H-pyran-4-yl, 1-methoxybutan-2-yl, 1-aminobutan-2-yl, and 1-morpholinobutan-2-yl;
provided that R₁ is not hex-1-enyl.

11: The method of claim 10, wherein R₁ is selected from the group consisting of butenyl, propenyl, vinyl, and ethynyl.

12. (canceled)

13: The method of claim 10, wherein R₁ is ethynyl.

14: The method of claim 10, wherein R₂ is selected from the group consisting of 3-pentyl, 4-heptyl, 4-methyl-1-morpholinopentan-2-yl, isobutyl, sec-butyl, tert-butyl, isopropyl, 1-hydroxybutan-2-yl, tetrahydro-2H-pyran-4-yl, 1-methoxybutan-2-yl, 1-aminobutan-2-yl, and 1-morpholinobutan-2-yl.

15. (canceled)

16: The method of claim 10, wherein R₂ is 3-pentyl.

17: The method of claim 10, wherein the compound of Formula A is selected from the group consisting of:
1-[(1R)-1-(morpholin-4-ylmethyl)propyl]-6-ethynylimidazo[4,5-b]pyrazin-2-ol;
(E)-1-(pentan-3-yl)-6-(prop-1-enyl)-1H-imidazo[4,5-b]pyrazin-2-ol;
(E)-1-cyclohexyl-6-(prop-1-enyl)-1H-imidazo[4,5-b]pyrazin-2-ol;
(E)-1-cyclopropyl-6-(prop-1-enyl)-1H-imidazo[4,5-b]pyrazin-2-ol;
(E)-1-isopropyl-6-(prop-1-enyl)-1H-imidazo[4,5-b]pyrazin-2-ol;
(E)-6-(prop-1-enyl)-1-(tetrahydro-2H-pyran-4-yl)-1H-imidazo[4,5-b]pyrazin-2-ol;
(Z)-1-(pentan-3-yl)-6-(prop-1-enyl)-1H-imidazo[4,5-b]pyrazin-2-ol;
(Z)-1-cyclohexyl-6-(prop-1-enyl)-1H-imidazo[4,5-b]pyrazin-2-ol;
(Z)-1-cyclopropyl-6-(prop-1-enyl)-1H-imidazo[4,5-b]pyrazin-2-ol;
(Z)-1-isopropyl-6-(prop-1-enyl)-1H-imidazo[4,5-b]pyrazin-2-ol;
(Z)-6-(prop-1-enyl)-1-(tetrahydro-2H-pyran-4-yl)-1H-imidazo[4,5-b]pyrazin-2-ol;
1-(pentan-3-yl)-6-(prop-1-ynyl)-1H-imidazo[4,5-b]pyrazin-2-ol;
6-ethynyl-1-(pentan-3-yl)-1H-imidazo[4,5-b]pyrazin-2-ol;
1-(ethylpropyl)-6-vinylimidazo[4,5-b]pyrazin-2-ol; and
1-(ethylpropyl)-6-(1-methylvinyl)imidazo[4,5-b]pyrazin-2-ol;
or a pharmaceutically acceptable salt thereof.

18: The method of claim 10, wherein the compound of Formula B is selected from the group consisting of:
(R)-6-ethynyl-1-(1-morpholinobutan-2-yl)-1H-imidazo[4,5-b]pyrazin-2(3H)-one;
(E)-1-(pentan-3-yl)-6-(prop-1-enyl)-1H-imidazo[4,5-b]pyrazin-2(3H)-one;
(E)-1-cyclohexyl-6-(prop-1-enyl)-1H-imidazo[4,5-b]pyrazin-2(3H)-one;
(E)-1-cyclopropyl-6-(prop-1-enyl)-1H-imidazo[4,5-b]pyrazin-2(3H)-one;
(E)-1-isopropyl-6-(prop-1-enyl)-1H-imidazo[4,5-b]pyrazin-2(3H)-one;
(E)-6-(prop-1-enyl)-1-(tetrahydro-2H-pyran-4-yl)-1H-imidazo[4,5-b]pyrazin-2(3H)-one;
(Z)-1-(pentan-3-yl)-6-(prop-1-enyl)-1H-imidazo[4,5-b]pyrazin-2(3H)-one;
(Z)-1-cyclohexyl-6-(prop-1-enyl)-1H-imidazo[4,5-b]pyrazin-2(3H)-one;
(Z)-1-cyclopropyl-6-(prop-1-enyl)-1H-imidazo[4,5-b]pyrazin-2(3H)-one;
(Z)-1-isopropyl-6-(prop-1-enyl)-1H-imidazo[4,5-b]pyrazin-2(3H)-one;
(Z)-6-(prop-1-enyl)-1-(tetrahydro-2H-pyran-4-yl)-1H-imidazo[4,5-b]pyrazin-2(3H)-one;
1-(pentan-3-yl)-6-(prop-1-ynyl)-1H-imidazo[4,5-b]pyrazin-2(3H)-one;
6-ethynyl-1-(pentan-3-yl)-1H-imidazo[4,5-b]pyrazin-2(3H)-one;
1-(pentan-3-yl)-6-vinyl-1H-imidazo[4,5-b]pyrazin-2(3H)-one; and
1-(pentan-3-yl)-6-(prop-1-en-2-yl)-1H-imidazo[4,5-b]pyrazin-2(3H)-one;
or a pharmaceutically acceptable salt thereof.

19: The method of claim 10, wherein the skeletal muscle troponin activator is
6-ethynyl-1-(pentan-3-yl)-1H-imidazo[4,5-b]pyrazin-2-ol, and
or a pharmaceutically acceptable salt thereof.

20: The method of claim 10, wherein the skeletal muscle troponin activator is 6-ethynyl-1-(pentan-3-yl)-1H-imidazo[4,5-b]pyrazin-2(3H)-one, or a pharmaceutically acceptable salt thereof.

21. (canceled)

22: The method of claim 1, wherein the skeletal muscle troponin activator is a compound of Formula I: 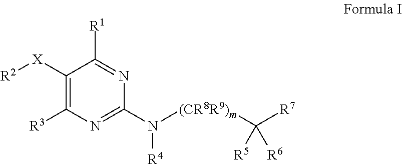
or a pharmaceutically acceptable salt thereof, wherein:
R¹ is selected from the group consisting of hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl, and 5-10 membered heteroaryl;
R² is selected from the group consisting of C₃₋₈ cycloalkyl, C₃₋₈ cycloalkenyl, 3-8 membered heterocycloalkyl, 3-8 membered heterocycloalkenyl, C₆₋₁₀ aryl, 5-10 membered heteroaryl and NR^{b}R^{c}, wherein each of the C₃₋₈ cycloalkyl, C₃₋₈ cycloalkenyl, 3-8 membered heterocycloalkyl, 3-8 membered heterocycloalkenyl, C₆₋₁₀ aryl, or 5-10 membered heteroaryl groups is optionally substituted with 1, 2, 3, 4, or 5 substituents selected from the group consisting of halogen, CN, oxo, (CH₂)ₙOR^{a}, (CH₂)ₙOC(O)R^{a}, (CH₂)ₙOC(O)OR^{a}, (CH₂)ₙOC(O)NR^{b}R^{c}, (CH₂)ₙNR^{b}R^{c}, (CH₂)ₙNR^{d}C(O)R^{a}, (CH₂)ₙNR^{d}C(O)OR^{a}, (CH₂)ₙNR^{d}C(O)NR^{b}R^{c}, (CH₂)NR^{d}C(O)C(O)NR^{b}R^{c}, (CH₂)ₙNR^{d}C(S)R^{a}, (CH₂)ₙNR^{d}C(S)OR^{a}, (CH₂)ₙNR^{d}C(S)NR^{b}R^{c}, (CH₂)ₙNR^{d}C(NR^{e})NR^{b}R^{c}, (CH₂)ₙNR^{d}S(O)R^{a}, (CH₂)ₙNR^{d}SO₂R^{a}, (CH₂)ₙNR^{d}SO₂NR^{b}R^{c}, (CH₂)ₙC(O)R^{a}, (CH₂)ₙC(O)OR^{a}, (CH₂)ₙC(O)NR^{b}R^{c}, (CH₂)C(S)R^{a}, (CH₂)C(S)OR^{a}, (CH₂)ₙC(S)NR^{b}R^{c}, (CH₂)ₙC(NR^{e})NR^{b}R^{c}, (CH₂)ₙSR^{a}, (CH₂)ₙS(O)R^{a}, (CH₂)ₙSO₂R^{a}, (CH₂)SO₂NR^{b}R^{c}, C₁₋₆ alkyl, C₁₋₆ haloalkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙC₆₋₁₀ aryl, and (CH₂)ₙ5-10 membered heteroaryl, wherein each of the C₁₋₆ alkyl, C₂₋₆ alkenyl, C₂₋₆ alkynyl, (CH₂)ₙC₃₋₈ cycloalkyl, (CH₂)ₙ3-8 membered heterocycloalkyl, (CH₂)ₙC₆₋₁₀ aryl, or (CH₂)ₙ5-10 membered heteroaryl groups is optionally substituted with 1, 2, 3, 4, or 5 R^{f} substituents;
R³ is selected from the group consisting of hydrogen, halogen, CN, C₁₋₆ alkyl, C₁₋₆ haloalkyl, C(O)OR^{a}, C(O)NR^{b}R^{c}, OR^{a}, NR^{b}R^{c}, C₆₋₁₀ aryl, and 5-10 membered heteroaryl;
R⁴ is selected from the group consisting of hydrogen, C₁₋₆ alkyl, C₁₋₆ haloalkyl, C(O)R^{a}, C(O)OR^{a}, C(O)NR^{b}R^{c}, and SO₂R^{a};
R⁵ and R⁶ are each independently selected from the group consisting of hydrogen, halogen, C₁₋₆ alkyl, and C₁₋₆ haloalkyl;
or alternatively, R⁵ and R⁶ together with the carbon atom to which they are bound form a group selected from the group consisting of C₃₋₈ cycloalkyl, C₃₋₈ cycloalkenyl, 3-8 membered heterocycloalkyl, and 3-8 membered heterocycloalkenyl, each optionally substituted with 1, 2, 3, 4, or 5 substituents selected from the group consisting of halogen, CN, oxo, OR^{a}, OC(O)R^{a}, OC(O)OR^{a}, NR^{b}R^{c}, C(O)R^{a}, C(O)OR^{a}, C(O)NR^{b}R^{c}, S(O)R^{a}, SO₂R^{a}, SO₂NR^{b}R^{c}, C₁₋₆ alkyl, and C₁₋₆ haloalkyl;
R⁷ is selected from the group consisting of C₃₋₈ cycloalkyl, C₃₋₈ cycloalkenyl, 3-8 membered heterocycloalkyl, 3-8 membered heterocycloalkenyl, C₆₋₁₀ aryl, and 5-10 membered heteroaryl, each optionally substituted with 1, 2, 3, 4, or 5 substituents selected from halogen, CN, oxo, OR^{a}, OC(O)R^{a}, OC(O)OR^{a}, OC(O)NR^{b}R^{c}, NR^{b}R^{c}, NR^{d}C(O)R^{a}, NR^{d}C(O)OR^{a}, NR^{d}C(O)NR^{b}R^{c}, NR^{d}C(O)C(O)NR^{b}R^{c}, NR^{d}C(S)R^{a}, NR^{d}C(S)OR^{a}, NR^{d}C(S)NR^{b}R^{c}, NR^{d}C(NR^{e})NR^{b}R^{c}, NR^{d}S(O)R^{a}, NR^{d}SO₂R^{a}, NR^{d}SO₂NR^{b}R^{c}, C(O)R^{a}, C(O)OR^{a}, C(O)NR^{b}R^{c}, C(S)R^{a}, C(S)OR^{a}, C(S)NR^{b}R^{c}, C(NR^{e})NR^{b}R^{c}, SR^{a}, S(O)R^{a}, SO₂R^{a}, SO₂NR^{b}R^{c}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, or 5-10 membered heteroaryl groups is optionally substituted with 1, 2, 3, 4, or 5 R^{f} substituents;
R⁸ and R⁹, at each occurrence, are each independently selected from the group consisting of hydrogen, halogen, and C₁₋₆ alkyl;
X is selected from the group consisting of a bond, —(CH₂)ₚ—, —(CH₂)ₚC(O)(CH₂)_{q}—, —(CH₂)ₚO(CH₂)_{q}—, —(CH₂)ₚS(CH₂)_{q}—, —(CH₂)ₚNR^{d}(CH₂)_{q}—, —(CH₂)ₚC(O)O(CH₂)_{q}—, —(CH₂)ₚOC(O)(CH₂)_{q}—, —(CH₂)ₚNR^{d}C(O)(CH₂)_{q}—, —(CH₂)ₚC(O)NR^{d}(CH₂)_{q}—, —(CH₂)ₚNR^{d}C(O)NR^{d}(CH₂)_{q}—, —(CH₂)ₚNR^{d}SO₂(CH₂)_{q}—, and —(CH₂)ₚSO₂NR^{d}(CH₂)_{q}—;
or alternatively, X, R², and R³, together with the carbon atoms to which they are bound, form a 5-6 membered ring optionally containing one or more heteroatoms selected from oxygen, nitrogen and sulfur, and optionally containing one or more double bonds, and optionally substituted with 1, 2, 3, 4, or 5 R^{f} substituents;
R^{a}, at each occurrence, is independently selected from the group consisting of hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, or 5-10 membered heteroaryl groups is optionally substituted with 1, 2, 3, 4, or 5 R^{f} substituents;
R^{b} and R^{c}, at each occurrence, are each independently selected from the group consisting of hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, 5-10 membered heteroaryl, C(O)R^{g}, C(O)OR^{g}, C(O)NRⁱR^{j}, and SO₂R^{g}, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, or 5-10 membered heteroaryl groups is optionally substituted with 1, 2, 3, 4, or 5 R^{f} substituents;
R^{d}, at each occurrence, is independently selected from the group consisting of hydrogen and C₁₋₆ alkyl;
R^{e}, at each occurrence, is independently selected from the group consisting of hydrogen, CN, OH, C₁₋₆ alkoxy, C₁₋₆ alkyl, and C₁₋₆ haloalkyl;
R^{f}, at each occurrence, is independently selected from the group consisting of halogen, CN, OR^{h}, OC(O)R^{h}, OC(O)OR^{h}, OC(O)NRⁱR^{j}, NRⁱR^{j}, NR^{d}C(O)R^{h}, NR^{d}C(O)OR^{h}, NR^{d}C(O)NRⁱR^{j}, NR^{d}C(O)C(O)NRⁱR^{j}, NR^{d}C(S)R^{h}, NR^{d}C(S)OR^{h}, NR^{d}C(S)NRⁱR^{j}, NR^{d}C(NR^{e})NRⁱR^{j}, NR^{d}S(O)R^{h}, NR^{d}SO₂R^{h}, NR^{d}SO₂NRⁱR^{j}, C(O)R^{h}, C(O)OR^{h}, C(O)NRⁱR^{j}, C(S)R^{h}, C(S)OR^{h}, C(S)NRⁱR^{j}, C(NR^{e})NRⁱR^{j}, SR^{h}, S(O)R^{h}, SO₂R^{h}, SO₂NRⁱR^{j}, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, or 5-10 membered heteroaryl groups is optionally substituted with 1, 2, 3, 4, or 5 R^{k} substituents;
or two R^{f} substituents bound to a single carbon atom, together with the carbon atom to which they are both bound, form a group selected from the group consisting of carbonyl, C₃₋₈ cycloalkyl, and 3-8 membered heterocycloalkyl;
R^{g}, at each occurrence, is independently selected from the group consisting of C₁₋₆ alkyl, C₁₋₆ haloalkyl, phenyl, naphthyl, and C₇₋₁₁ aralkyl, each optionally substituted with 1, 2, 3, 4, or 5 substituents selected from the group consisting of halogen, CN, OH, C₁₋₆ alkoxy, C₁₋₆ alkyl, and C₁₋₆ haloalkyl;
R^{h}, at each occurrence, is independently selected from the group consisting of hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, and 5-10 membered heteroaryl, wherein each of the C₁₋₆ alkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, or 5-10 membered heteroaryl groups is optionally substituted with 1, 2, 3, 4, or 5 R^{k} substituents;
Rⁱ and R^{j}, at each occurrence, are each independently selected from the group consisting of hydrogen, C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, 5-10 membered heteroaryl, C(O)R^{g}, and C(O)OR^{g}, wherein each of the C₁₋₆ alkyl, C₁₋₆ haloalkyl, C₂₋₆ alkenyl, C₂₋₆ alkynyl, C₃₋₈ cycloalkyl, C₃₋₈ cycloalkenyl, 3-8 membered heterocycloalkyl, 3-8 membered heterocycloalkenyl, C₆₋₁₀ aryl, C₇₋₁₁ aralkyl, or 5-10 membered heteroaryl groups is optionally substituted with 1, 2, 3, 4, or 5 substituents selected from the group consisting of halogen, CN, OH, C₁₋₆ alkoxy, C₁₋₆ alkyl, and C₁₋₆ haloalkyl;
R^{k}, at each occurrence, is independently selected from the group consisting of halogen, CN, OH, C₁₋₆ alkoxy, NH₂, NH(C₁₋₆ alkyl), N(C₁₋₆ alkyl)₂, NHC(O)C₁₋₆ alkyl, NHC(O)C₇₋₁₁ aralkyl, NHC(O)OC₁₋₆ alkyl, NHC(O)OC₇₋₁₁ aralkyl, OC(O)C₁₋₆ alkyl, OC(O)C₇₋₁₁ aralkyl, OC(O)OC₁₋₆ alkyl, OC(O)OC₇₋₁₁ aralkyl, C(O)C₁₋₆ alkyl, C(O)C₇₋₁₁ aralkyl, C(O)OC₁₋₆ alkyl, C(O)OC₇₋₁₁ aralkyl, C₁₋₆ alkyl, C₁₋₆ haloalkyl, C₂₋₆ alkenyl, and C₂₋₆ alkynyl, wherein each C₁₋₆ alkyl, C₂₋₆ alkenyl, C₂₋₆ alkynyl, or C₇₋₁₁ aralkyl substituent is optionally substituted with 1, 2, or 3 substituents selected from the group consisting of OH, C₁₋₆ alkoxy, NH₂, NH(C₁₋₆ alkyl), N(C₁₋₆ alkyl)₂, NHC(O)C₁₋₆ alkyl, NHC(O)C₇₋₁₁ aralkyl, NHC(O)OC₁₋₆ alkyl, and NHC(O)OC₇₋₁₁ aralkyl;
or two R^{k} substituents bound to a single carbon atom, together with the carbon atom to which they are both bound, form a carbonyl group;
m is 0, 1 or 2;
n, at each occurrence, independently is 0, 1, or 2;
p is 0, 1, or 2; and
q is 0, 1, or 2.

23: The method of claim 22, wherein the skeletal muscle troponin activator is a compound of Formula V(a) or V(b), or a pharmaceutically acceptable salt thereof: 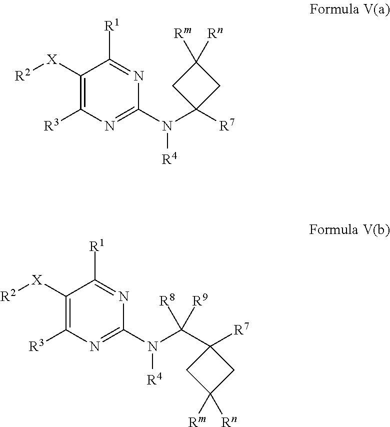
wherein R^{m} and Rⁿ are each independently selected from the group consisting of hydrogen, halogen, and C₁₋₆ alkyl.

24. (canceled)

25: The method of claim 22, wherein the skeletal muscle troponin activator is a compound of Formula XII(a), or a pharmaceutically acceptable salt thereof:

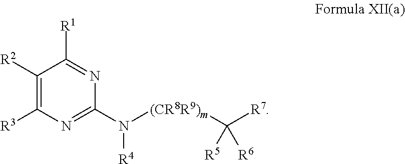

26: The method of claim 22, wherein the skeletal muscle troponin activator is
1-(2-(((trans)-3-fluoro-1-(3-fluoropyridin-2-yl)cyclobutyl)methylamino)pyrimidin-5-yl)-1H-pyrrole-3-carboxamide, wherein
1-(2-(((trans)-3-fluoro-1-(3-fluoropyridin-2-yl)cyclobutyl)methylamino)pyrimidin-5-yl)-1H-pyrrole-3-carboxamide is defined by the following structure: 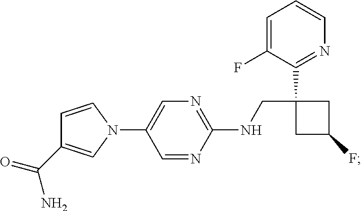 [SMILES: NC(=O)c1ccn(-c2cnc(NC[C@]3(c4ncccc4F)C[C@H](F)C3)nc2)c1]
or a pharmaceutically acceptable salt thereof.

27. (canceled)

28: The method of claim 10, wherein the patient suffers from a disease or condition selected from the group consisting of sleep-disordered breathing, diaphragmatic atrophy, ventilator-induced diaphragmatic weakness or atrophy, steroid-induced diaphragmatic atrophy, hemidiaphragm paralysis, fetal hydrops, pleural effusion, botulinum poisoning, organophosphate poisoning, Guillain-Barre syndrome, phrenic nerve dysfunction, stroke, asthma, Arnold-Chiari malformation, quadriplegia, poliomyelitis, syringomyelia, tumor compression, idiopathic hyperinflation, botulism, drug use, and acid maltase deficiency.

29: The method of claim 22, wherein the patient suffers from a disease or condition selected from the group consisting of sleep-disordered breathing, ventilator-induced diaphragmatic weakness or atrophy, steroid-induced diaphragmatic atrophy, hemidiaphragm paralysis, fetal hydrops, pleural effusion, botulinum poisoning, organophosphate poisoning, phrenic nerve dysfunction, asthma, Arnold-Chiari malformation, quadriplegia, poliomyelitis, syringomyelia, tumor compression, idiopathic hyperinflation, botulism, and acid maltase deficiency.

30: The method of claim 1, wherein the skeletal muscle troponin activator is a compound of Formula A or a compound of Formula B: 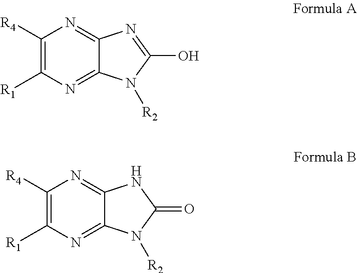
or a pharmaceutically acceptable salt thereof, wherein:
R₁ and R₄ are independently selected from the group consisting of hydrogen, halo, hydroxy, optionally substituted acyl, optionally substituted alkyl, optionally substituted amino, optionally substituted alkenyl, optionally substituted alkynyl, optionally substituted cycloalkyl, optionally substituted heteroaryl, optionally substituted alkoxy, optionally substituted aminocarbonyl, sulfonyl, sulfanyl, sulfinyl, carboxy, optionally substituted alkoxycarbonyl, and cyano; or
R₄ and R₁, taken together with any intervening atoms, form a fused ring system selected from the group consisting of optionally substituted fused aryl, optionally substituted fused heteroaryl, optionally substituted fused cycloalkyl, and optionally substituted fused heterocycloalkyl; and
R₂ is selected from the group consisting of optionally substituted alkyl, optionally substituted cycloalkyl, optionally substituted aryl, optionally substituted heteroaryl, and optionally substituted heterocycloalkyl;
provided that R₁ is not hex-1-enyl; and
further provided that
the compound of Formula A or the compound of Formula B is not
(S)-6-bromo-1-(1-phenylethyl)-1H-imidazo[4,5-b]pyrazin-2(3H)-one;
1,5,6-trimethyl-1H-imidazo[4,5-b]pyrazin-2(3H)-one;
1-methyl-1H-imidazo[4,5-b]pyrazin-2(3H)-one;
6-bromo-1-(3-nitrobenzyl)-1H-imidazo[4,5-b]pyrazin-2(3H)-one;
5-(hydroxymethyl)-1,6-dimethyl-1H-imidazo[4,5-b]pyrazin-2(3H)-one; or
1-(piperidin-4-yl)-1H-imidazo[4,5-b]pyrazin-2(3H)-one.
